(19) United States
(12) Patent Application Publication
(10) Pub. No.: US 2011/0238143 A1
(43) Pub. Date: Sep. 29, 2011

(54) SYSTEM AND METHOD FOR ALTERING AND MAINTAINING THE BODY TEMPERATURE OF A PATIENT

(75) Inventors: Robert B. Schock, Sparta, NJ (US); Robert J. Freedman, JR., Alexandria, LA (US)

(73) Assignee: Life Recovery Systems HD, LLC, Waldwick, NJ (US)

(21) Appl. No.: 13/057,664

(22) PCT Filed: Aug. 5, 2009
(86) PCT No.: PCT/US09/52838

§ 371 Date: Jun. 9, 2011

Related U.S. Application Data
(60) Provisional application No. 61/087,044, filed on Aug. 7, 2008.

(51) Int. Cl. A61F 7/00

(52) U.S. Cl. 607/104

(57) ABSTRACT

A system includes a temperature reducing apparatus having a first enclosure defining an interior space for receiving at least a portion of a patient's body therein, and a liquid delivery system in fluid communication with the first enclosure for controlling the temperature of a heat transfer liquid and delivering the heat transfer liquid into the first enclosure in direct contact with the patient's body portion when received in the first enclosure. A temperature maintenance apparatus includes a second enclosure defining an interior space for receiving at least a portion of a patient's body therein, and a gas delivery system in fluid communication with the second enclosure for controlling the temperature of a heat transfer gas and delivering the heat transfer gas into the second enclosure in direct contact with the patient's body portion when received in the second enclosure. The second enclosure is different than the first enclosure.

### BACKGROUND OF THE INVENTION

This invention generally relates to medical systems and methods for altering the body temperature of a patient and more particularly to systems and methods that enable efficient, quick adjustment of the body temperature of a patient, especially to induce hypothermia.

Sudden cardiac arrest remains a serious public health issue. Approximately 350,000 individuals are stricken in the United States annually, with overall survival rates of roughly 5 percent. Even with the immediate availability of the most advanced care currently available, including cardiopulmonary resuscitation (CPR), drugs, ventilation equipment, and automatic external defibrillators, a survival rate of 25 percent may be the probable best case scenario. Improved therapies to deal with this condition are clearly needed.

Numerous incidences of recovery following accidental hypothermia and cardiac arrest have been reported. This observation has led researchers to consider therapeutic hypothermia as a possible treatment for reducing the adverse consequences of circulatory arrest. Various studies have shown that moderate systemic hypothermia (approximately 3-5° C. (5.4-9.0° F.)) can reduce damage to vital organs, including the brain. Hypothermia induced both during and following cardiac arrest has demonstrated this benefit. The use of cardiopulmonary bypass has also been effective in rapidly achieving this goal. Direct flushing of cooled fluids into the arterial or venous system has also been employed with success. Both invasive measures, however, require large bore intravascular catheters and rapid introduction of sterile solutions into the patient. Such invasive approaches have obvious disadvantages in dealing with out-of-hospital emergencies.

Noninvasive cooling, if sufficiently effective and portable, would be a preferable approach. Direct cooling of the head alone has produced variable results. However, post-resuscitative cooling of the entire body to approximately 33° C. (91.4° F.) by noninvasive treatment has been demonstrated to be surprisingly effective in recent clinical studies. The use of cold gel and ice packs produced cooling of approximately 0.9° C. (1.6° F.) per hour, and resulted in a nearly 100 percent improvement in neurologically intact survival (Bernard S. A. et al., Treatment of Comatose Survivors of Out-of-Hospital Cardiac Arrest with Induced Hypothermia, 346 NEW ENG. J. MED. 557-563 (2002)). In another study, cold air was found to be capable of cooling patients at a rate of about 0.25° C. (0.45° F.) per hour, which caused a 40 percent improvement in the same endpoint (Sterz F et al., Mild Therapeutic Hypothermia to Improve the Neurologic Outcome after Cardiac Arrest, 346 NEW ENG. J. MED. 549-556 (2002)). In yet another study, a combination of water-filled cooling blankets and ice packs applied to the skin resulted in a cooling rate of 0.8° C. (1.4° F.) per hour (Felberg et al., Hypothermia After Cardiac Arrest—Feasibility and Safety of an External Cooling Protocol, 104 CIRCULATION 1799-1804 (2001)). In still another study, inducing hypothermia and maintaining the patient in that condition for an extended period of time (e.g., several days) followed by gradual rewarming (0.2° C./hr to 0.5° C./hr) has been shown to be beneficial to the patient (Polderman, Induced Hypothermia and Fever Control for Prevention and Treatment of Neurological Injuries, The Lancet, Vol. 371, 1955-69 (Jun. 7, 2008)).

It is believed that increasing the rate of cooling from what is shown in these studies and improved patient management may produce a higher rate of patient salvage.

### SUMMARY OF THE INVENTION

In one aspect, a system generally comprises a temperature reducing apparatus comprising a first enclosure defining an interior space for receiving at least a portion of a patient's body therein, and a liquid delivery system in fluid communication with the first enclosure for controlling the temperature of a heat transfer liquid and delivering the heat transfer liquid into the first enclosure in direct contact with the patient's body portion when received in the first enclosure. A temperature maintenance apparatus of the system comprises a second enclosure defining an interior space for receiving at least a portion of a patient's body therein, and a gas delivery system in fluid communication with the second enclosure for controlling the temperature of a heat transfer gas and delivering the heat transfer gas into the second enclosure in direct contact with the patient's body portion when received in the second enclosure, the second enclosure being different than the first enclosure.

In another aspect, a system generally comprises a temperature reducing apparatus comprising a first enclosure defining an interior space for receiving at least a portion of a patient's body therein, and a liquid delivery system in fluid communication with the first enclosure for controlling the temperature of a heat transfer liquid and delivering the heat transfer liquid into the first enclosure in direct contact with the patient's body portion when received in the first enclosure. A temperature maintenance and re-warming apparatus of the system generally comprises a second enclosure defining an interior space for receiving at least a portion of a patient's body therein, and a gas delivery system in fluid communication with the second enclosure for controlling the temperature of a heat transfer gas and delivering the heat transfer gas into the second enclosure in direct contact with the patient's body portion when received in the second enclosure. The delivery system has a heat exchanger capable of warming the heat transfer gas and cooling the heat transfer gas.

In yet another aspect, a method for operating a system adapted to adjust the body temperature of a patient generally comprises enclosing at least a portion of a patient's body within an interior space of a first enclosure. The first enclosure has an inlet for receiving a heat transfer liquid into the interior space and an outlet in fluid communication with the interior space of the enclosure for exhausting the heat transfer liquid from the enclosure. The heat transfer liquid is directed through the inlet of the enclosure into the interior space for flow over the patient's body in direct liquid contact therewith to promote heat transfer between the patient's body and the heat transfer liquid to the outlet of the enclosure. The portion of the patient's body is removed from the first enclosure and enclosed within an interior space of a second enclosure. A heat transfer gas is directed into the interior space of the second enclosure for flow over the patient's body in direct contact therewith to promote heat transfer between the patient's body and the heat transfer gas.

In still another aspect, a gas body temperature cooling/heating apparatus generally comprises an enclosure defining an interior space for receiving at least a portion of a patient's body. The enclosure is adapted to allow heat transfer gas to flow into the interior space for direct contact with the patient's body to promote heat transfer between the patient and the heat transfer gas. A gas delivery system has a gas heat exchanger for controlling the temperature of the heat transfer gas, and an exhaust pump for drawing heat transfer gas from the interior space of the enclosure and creating a vacuum within the interior space of the enclosure.

Other objects and features will be in part apparent and in part pointed out hereinafter.

### BRIEF DESCRIPTION OF THE DRAWINGS

FIG. 1 is a perspective of an apparatus for altering the body temperature of a patient, the apparatus being positioned on a gurney;

FIG. 2 is a top plan of the apparatus removed from the gurney;

FIG. 3 is a section of the apparatus along line 3-3 of FIG. 2;

FIG. 4 is an exploded perspective of the apparatus;

FIG. 5 is a top plan of the apparatus with a cover pulled back and a porous layer partially cut away thereby exposing a passage;

FIG. 6 is a section along line 6-6 of FIG. 2 but with the patient removed;

FIG. 7 is an enlargement of a passage formed in the cover shown in FIG. 6;

FIG. 8 is an enlargement of the passage in a compliant support shown in FIG. 7;

FIG. 9 is an end elevation of the apparatus showing a flow restrictor;

FIG. 10 is a schematic of a control system of the apparatus;

FIG. 10A is a view of a display of the control system of FIG. 10;

FIG. 11 is a schematic of the apparatus showing an air pump pumping air into the compliant support;

FIG. 12 is a schematic of the apparatus showing two inlet pumps pumping heat transfer liquid into an interior space of the apparatus from the bottom and top;

FIG. 13 is a schematic of the apparatus showing the heat transfer liquid being exhausted from the interior space of the apparatus;

FIG. 14 is a schematic of the apparatus showing the air pump pumping heat transfer gas into the interior space of the apparatus;

FIG. 15 is a top plan of the apparatus with the cover pulled back to show another configuration of the compliant support;

FIG. 16 is a top plan of the apparatus with the cover pulled back to show yet another configuration of the compliant support;

FIG. 17 is a section similar to the one shown in FIG. 6 but with a different tube configuration;

FIG. 18 is a perspective of a system for altering and maintaining the body temperature of a patient;

FIG. 19 is an exploded perspective of a gas cooling/heating apparatus of the system of FIG. 18; and

FIG. 20 is a cross-section of the gas cooling/heating apparatus of FIG. 19 with a patient received in the apparatus.

Corresponding reference characters indicate corresponding parts throughout the several views of the drawings.

### DETAILED DESCRIPTION OF THE PREFERRED EMBODIMENT

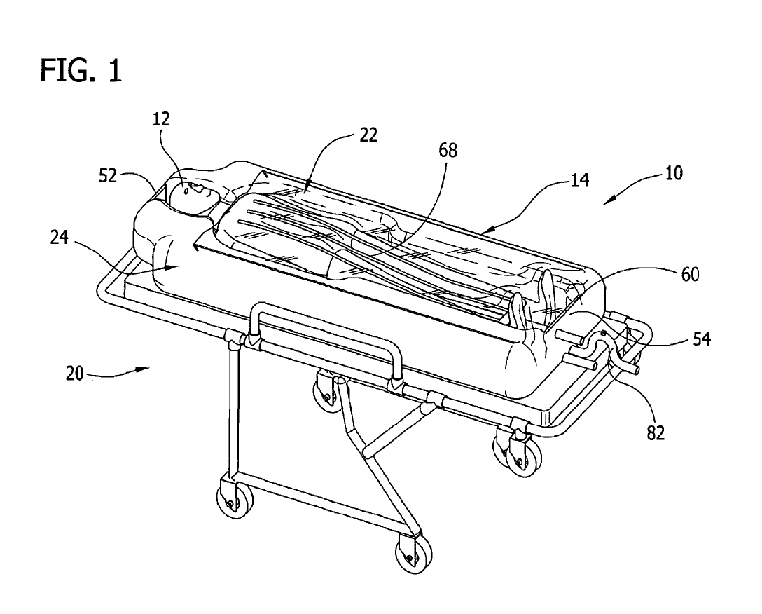

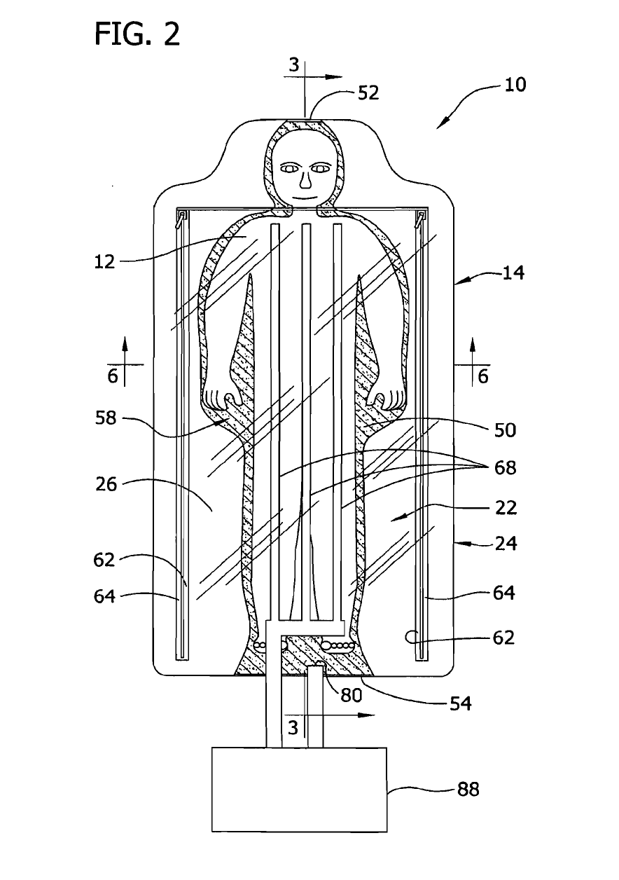

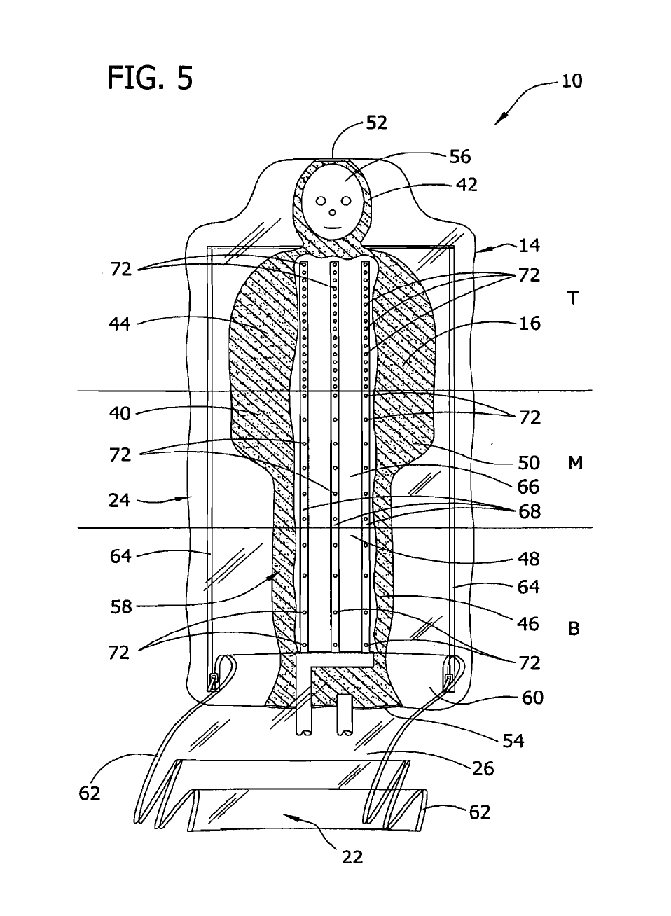

Referring now to the drawings and particularly to FIGS. 1 and 2, reference number 10 generally indicates an apparatus for adjusting the body temperature of a patient 12. The apparatus 10 generally comprises an enclosure, indicated at 14, defining an interior space 16 (FIG. 5) for receiving at least a portion of a patient's body. While it is understood that any portion of the patient's body (including the entire body) may be placed inside the enclosure 14, for exemplary purposes, the illustrated portion of the patient's body received in the interior space 16 of the enclosure 14 is the patient's body from the neck downward, including the torso, arms, and legs. The enclosure 14 is adapted to generally conform to the shape of the patient's body received therein to accommodate patients of various shapes and sizes. For example, in one configuration, the enclosure 14 is suitable for individuals having a size between about the 5th percentile and about the 95th percentile adult male. Enclosures adapted to receive smaller individuals (e.g., babies, children, small adults) or larger individuals are also contemplated.

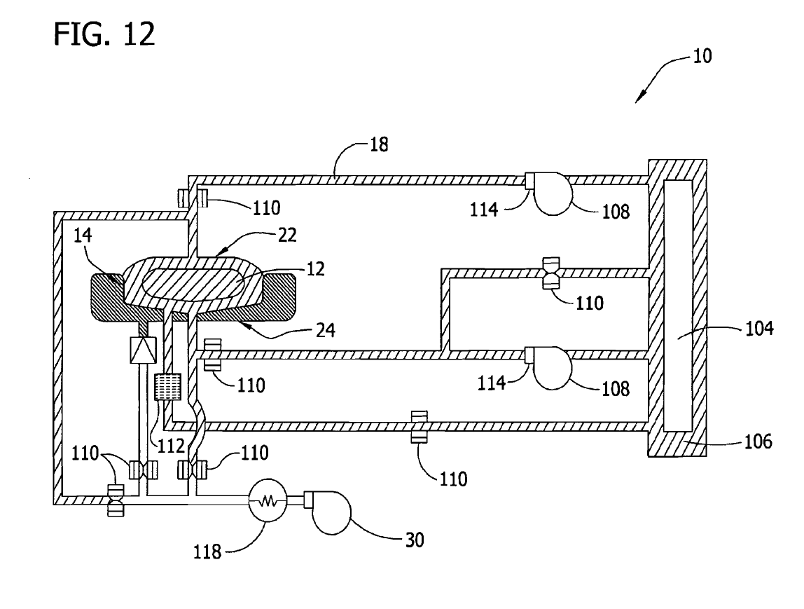

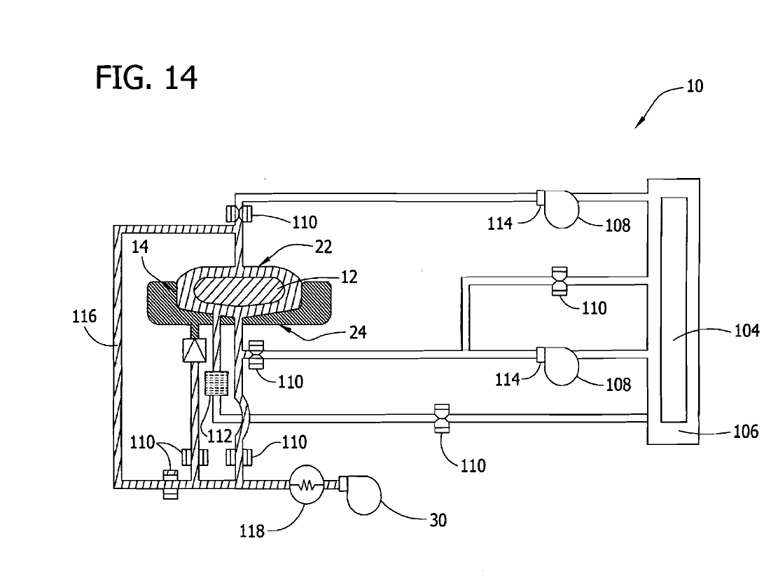

The enclosure 14 is also adapted to allow heat transfer liquid 18 (FIG. 12), such as water, saline or other suitable liquids, or heat transfer gas 116 (FIG. 14) to flow into the interior space 16 for direct contact with the patient's body to promote heat transfer between the patient 12 and the heat transfer fluid. To raise the temperature of a patient 12, the heat transfer fluid is directed into the interior space 16 of the enclosure 14 at a temperature greater than the temperature of the portion of the patient's body. For example, the heat transfer fluid may have a temperature in a range of about 37° C. (98.6° F.) to about 47° C. (117° F.), such as about 45° C. (113° F.). One application of such a warming enclosure would be to warm a patient 12 suffering from unintended hypothermia.

To lower the temperature of a patient 12, the heat transfer fluid is directed into the enclosure 14 at a temperature lower than the temperature of the body portion of the patient received in the interior space 16 of the enclosure so that the fluid cools the body portion of the patient. For example, the heat transfer fluid may have a temperature in a range of about 0.5° C. (34° F.) to about 4° C. (36° F.). Heat transfer fluid introduced into the enclosure 14 at such a temperature has been found to cool the body at a sufficient rate to induce hypothermia while minimizing any adverse effects to the skin of the patient. It is to be understood that temperatures other than those listed above can be used to adjust the temperature of a patient 12 received in the interior space 16 of the enclosure 14.

As mentioned above, hypothermia can be used to minimize or prevent damage to vital organs, including the brain, caused by cardiac arrest. It is well recognized that organ damage can, and typically does, occur shortly after the victim has suffered cardiac arrest. As a result, it is often in the victim's best interest to quickly and effectively induce hypothermia to minimize or prevent organ damage. Since many victims of cardiac arrest are initially treated by first responders (i.e., police officers, firefighters, emergency medical technicians), in one configuration, the apparatus 10 is portable for use remote from a medical facility. Moreover, the enclosure 14 is sized and shaped for placement on a stretcher, such as an ambulance or emergency gurney (generally indicated at 20), to facilitate the transportation of the patient 12 to a medical facility in a conventional manner while placed in the enclosure (FIG. 1). Accordingly, the enclosure 14 may have a width between about 66 centimeters (26 inches) and about 76 centimeters (30 inches) and a length between about 203 centimeters (80 inches) and about 210 centimeters (83 inches), the approximate range of dimensions for a standard ambulance or emergency gurney 20. It is contemplated that the enclosure 14 may have other configurations without departing from the scope of this invention. It is also contemplated that the enclosure 14 may be used to treat other medical conditions or have application in other medical procedures (e.g., hyperthermia, trauma, stroke, heart attack, enhancements of anti-cancer therapies, surgical support, spinal injury, and general thermal management). Moreover, although the patient 12 is most commonly a human, the apparatus 10 could be used for other animals.

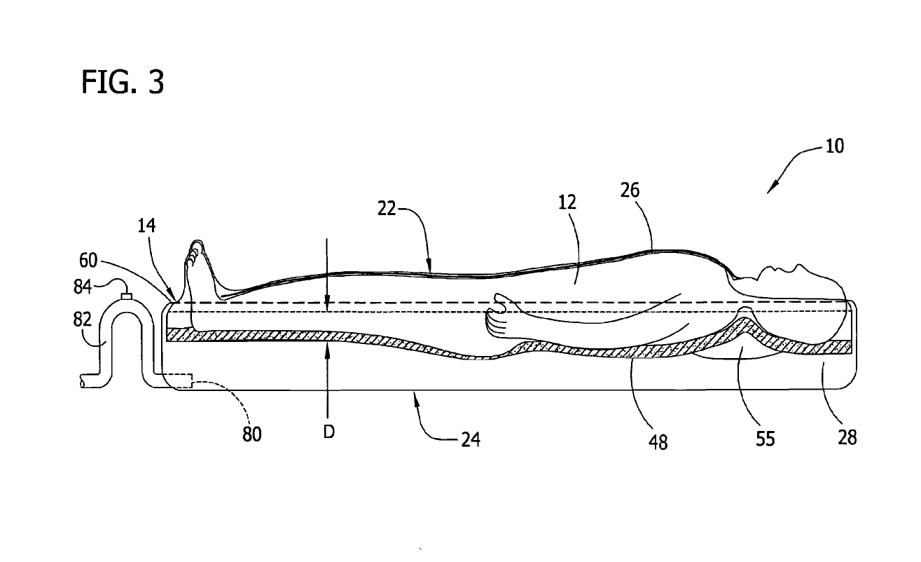

The amount of time necessary to induce hypothermia in a patient 12 is dependent on numerous factors including the portion of the patient received in the interior space 16 of the enclosure 14, the temperature of the heat transfer fluid, and the amount of time the heat transfer fluid is in contact with the portion of the patient's body. As a result, in one configuration, the enclosure 14 is adapted to enclose the patient's body from the neck down thereby providing a large portion of the patient's total surface area for heat transfer with the heat transfer fluid. As illustrated in FIG. 3, the enclosure 14 comprises a cover, indicated at 22, for overlying the patient 12 from the neck downward, and a compliant support, indicated at 24, for underlying the patient's entire body.

As shown in FIGS. 2 and 3, the cover 22 comprises a limp sheet-like member 26 adapted to generally conform, under its own weight, to the contours of the patient 12 which it is covering. The sheet-like member 26 is preferably made of a transparent material such as polyvinyl chloride (PVC), polyethylene, or polyurethane so that the body of the patient received within the enclosure can be viewed. It is understood that a sheet-like member (not shown) may be made of a non-transparent material or has a portion that is transparent with the remainder of sheet-like member being non-transparent. In another configuration (not shown), the cover 22 further comprises a porous layer disposed between the sheet-like member 26 and patient's body. The porous layer, such as batting or open-celled foam, supports the sheet-like member 26 in a position spaced from the patient's body thereby providing a fluid passageway for allowing the heat transfer fluid to pass between the sheet-like member and the patient's body.

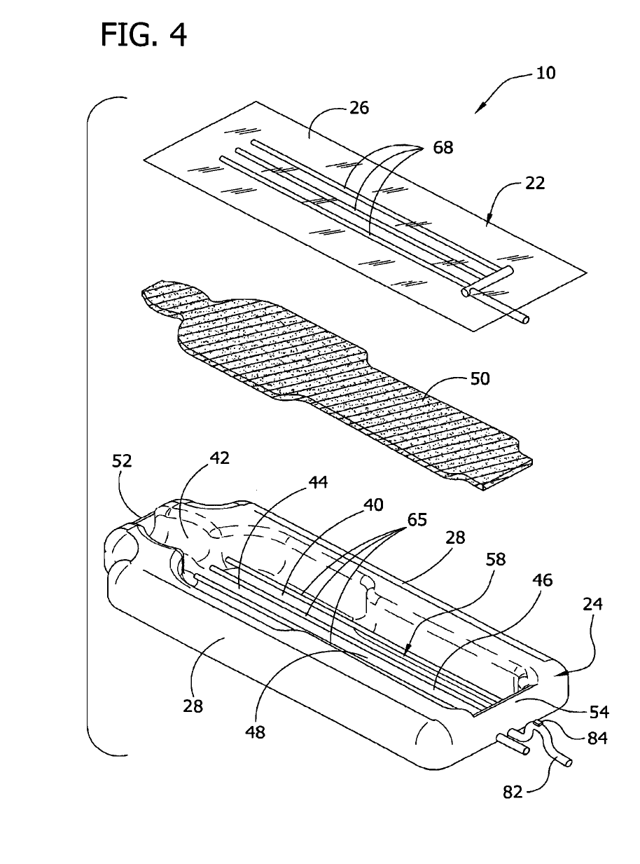

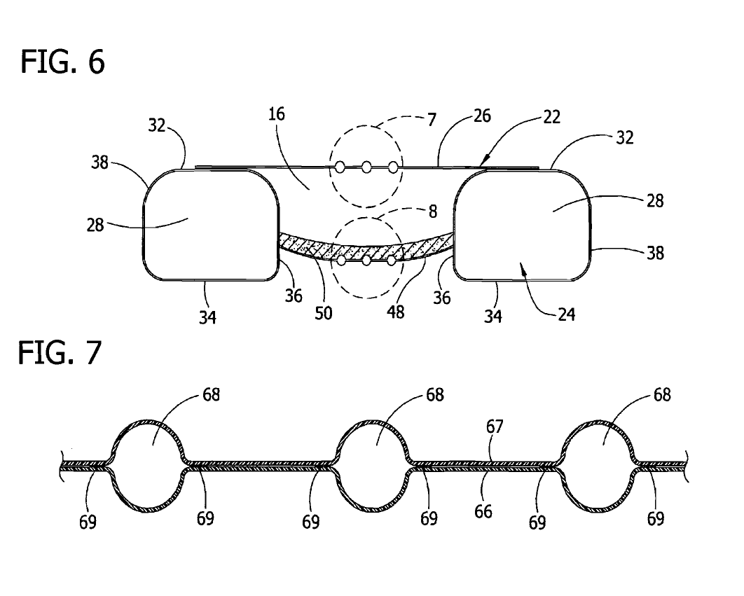

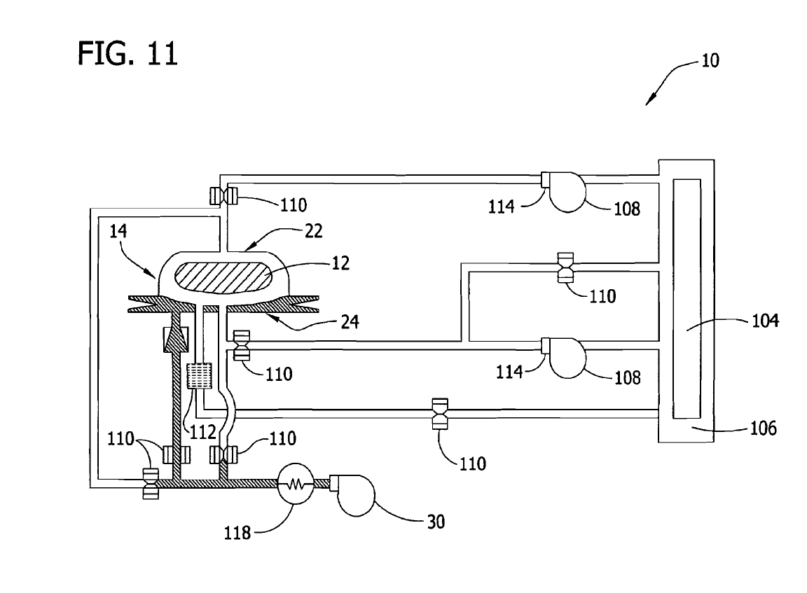

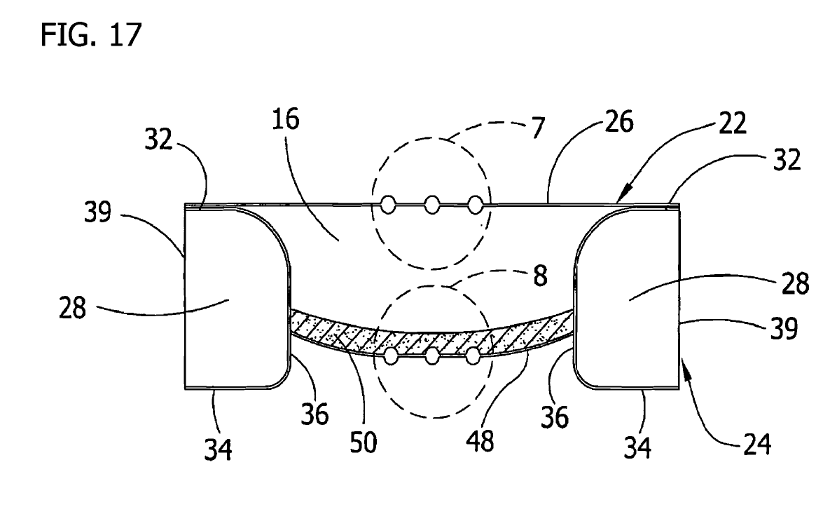

The compliant support 24 is a pneumatic support, which, like the cover 22, also generally conforms to the shape of the patient's body when the body rests on the support. Moreover, the compliant support 24 minimizes pressure concentrations beneath the patient 12 which facilitates flow of heat transfer fluid beneath the patient and minimizes the possibility of pressure sores developing in the skin of the patient. The compliant support 24, as illustrated in FIG. 4, comprises two generally elongate, gas-filled tubes 28, which form a right side and a left side of the support. As illustrated in FIG. 11, the gas-filled 28 tubes are selectively inflatable using an air pump 30 (or manually) and deflatable by bleeding the air from the tubes. Referring now to FIG. 6, each of the tubes 28 has a top 32, a bottom 34, an interior facing side 36, and an exterior facing side 38. The interior facing sides 38 are generally shaped to conform to the side profile of the patient 12. Accordingly, the tubes 28, when inflated, collectively define a recess 40 in a center of the support 24 (i.e., between the gas-filled tubes 28) for receiving the patient's body (FIG. 4). More specifically, the tubes 28 generally conform to the sides of the patient 12 as they are being inflated thereby forming a pocket 42 sized and shaped for receiving the head and neck of the patient, a broader region 44 for receiving the torso of the patient, and a tapered pocket 46 for receiving the legs and feet of the patient. The pocket 42, which is adapted for receiving the head and neck of the patient 12, is configured to support the head in an upward-facing direction thereby maintaining the patient's breathing passageways (i.e., nose and mouth) out of contact with the heat transfer fluid in the interior space 16 of the enclosure 14. The pocket 42 prevents the patient's head from moving to a side-facing direction. The tubes 28 may be captured on their exterior facing sides 38 (to define the outer perimeter of the support) by a restraint 39 made of a rigid material, such as a rigid plastic, to thereby limit outward expansion of the tubes (FIG. 17). The restraint 39 shown in FIG. 17 is integral with the support 24 (i.e., forms one wall of the support). However, it is contemplated that the restraint may be formed as a separate component. In another configuration (not shown), the restraint 39 may be a tether or a plurality of tethers extending between the interior walls of the tubes 28. The restraint 39 provides added support to the tubes which allows the tubes 28 to hold their shape under loads from the heat transfer liquid 18 and the patient's body weight. It is understood that the compliant support 24 may have different shapes and sizes or be conformable with the patient's body in a way different from that described herein. For example, the compliant support 24 could be filled with any suitable fluid, including a liquid, or any suitable flowable material, such as polystyrene beads.

With reference to FIGS. 4 and 6, a liquid impermeable sheet-like member 48 extends between the generally opposing portions of the tubes 28, and a porous layer 50 overlies the member. The impermeable member 48 is attached to the tubes 28 such that the member slopes from the head pocket 42 of the enclosure 14 toward the tapered foot pocket 46. The impermeable member 48 retains the heat transfer fluid within the enclosure 14, while a porous layer 50, such as rich loft polyester batting or an open-cell polyurethane foam, allows heat transfer fluid to pass into contact with the patient's body portion for flow across the skin throughout the enclosure. The impermeable member 48 comprises a transparent material such as PVC, polyethylene, or polyurethane. It is understood that the impermeable member 48 may comprise in its entirety a non-transparent material or have a portion that is transparent with the remainder of impermeable member being non-transparent. It is also understood that the impermeable member 48 may be attached to the tubes such that the impermeable member lies in a generally horizontal plane or is sloped from the foot pocket 46 toward the head pocket 42.

Referring again to FIG. 4, a front end panel 52 and a rear end panel 54 extend between the tubes 28 and define the forward and rearward extent of the support 24. The end panels 52, 54 can be made for a variety of materials, such as semi-rigid plastic, plastic foam, elastic plastic sheeting, an inflatable section, or a constrained inflatable section (e.g., a series of inflatable tubes bonded to one another).

The compliant support 24 further comprises a positioner 56 (FIG. 5), such as a head rest, a forehead strap, or indicia printed on the support, that indicates to the user where to properly place the patient 12 on the compliant support. The indicia may be text (such as written instructions), an outline of a body or portion thereof, or an image, such as an image of a face. It is contemplated that the positioner 56 may be placed anywhere on or in the enclosure.

As illustrated in FIG. 3, the compliant support 24 may further comprise a selectively inflatable head rest 55, which not only provides the user with an indication where to position the patient 12, but also maintains the patient's breathing passageways (i.e., nose and mouth) in spaced relation with the heat transfer fluid in the interior space 16 of the enclosure 14. Moreover, the head rest 55 angles the patient's head back thereby opening the patient's breathing passageways. Thus, the head rest 55 places the patient's head in a position suitable for mouth-to-mouth resuscitation, one of the steps in performing CPR. In another configuration (not shown), the head rest 55 provides a pillow for the patient's head to rest. As a result, the patient's head is angled forward which may necessitate the use of a breathing tube to assist the patient with breathing. Thus, the patient's head can be positioned generally flat, angled forward, or angled back thereby providing the user the option to select the position best suited for a particular patient 12. It is understood that the head rest 55 may be formed from a non-inflatable component. It is also understood that the head rest 55 may be integral with the compliant support 24 or formed as a separate component.

The end panels 52, 54, impermeable member 48, and gas-filled tubes 28 of the compliant support 24 are collectively configured to form a watertight well, generally indicated at 58, in the center of the compliant support for receiving the entire body of the patient 12. The patient 12 is positioned in a supine position on the impermeable member 48 with the tubes 28 in a deflated state. The tubes 28 are then inflated to conform the interior side walls 36 of the tubes to the portion of the patient's body juxtaposed thereto. The tubes 28 provide longitudinally extending walls to prevent heat transfer fluid 18 from leaking in a lateral direction. The end panels 52, 54 prevent the heat transfer fluid 18 from leaking in a longitudinal direction, and the impermeable member prevents the heat transfer fluid from leaking in the downward direction. It is understood that the impermeable member may also extend over the tubes and end panels thereby preventing leaking in all directions.

As a result, the well 58 is sized and spaced to generally conform to the patient's body received in the interior space 16 of the enclosure 14. Thus, the volume of heat transfer fluid necessary to effectively alter the temperature of the patient 12 is also dependent on the size and shape of the patient. For example, a larger patient will require more heat transfer fluid than will a smaller patient to achieve a similar rate of heat transfer. Moreover, the heat transfer fluid within the interior space 16 of the enclosure 14 is maintained in a relatively thin layer and near or in contact with the patient's body positioned the well 58. As a result, the amount of heat transfer liquid 18 necessary to effectively alter the temperature of the patient 12 can be minimized. This becomes increasingly important in remote areas where volumes of heat transfer liquid 18, which can become heavy, need to be carried by hand. For example, about 16 liters (4.2 gallons) of heat transfer liquid 18 would weigh about 16 kilograms (35 pounds) where as about 12 liters of heat transfer liquid would weigh about 12 kilograms (27 pounds).

The well 58 enables heat transfer between the underside and side of the patient's body by allowing heat transfer fluid 18 to accumulate under and adjacent to the patient's body, and allowing heat transfer fluid to be delivered to the patient's body from a location beneath the patient 12. The depth D of the well 58 is varied along a longitudinal axis of the enclosure (FIG. 3). The well 58 is deeper in the region receiving the torso of the patient 12 than in the regions receiving the head, legs, and feet since a large portion of the patient's weight is contained in the torso. The well 58 has a depth D between about 2.5 centimeters (1 inch) and about 20 centimeters (8 inches), and preferably between about 12.7 centimeters (5 inches) and about 15 centimeters (6 inches) in the region adapted to receive the torso. These depths correspond generally to about one-half of the chest heights of adult males between the 5th percentile and 95th percentile. This variation in well 58 depths allows more heat transfer liquid to accumulate around the torso of the patient, a region of the body amenable to heat transfer, than with the head, legs, and feet of the patient 12. The reasons for managing the depth of the heat transfer liquid 18 in the region adapted to receive the head of the patient are apparent. It is understood that the well 58 can have a generally uniform depth D or have depths different from those indicated, such as when the enclosure 14 is designed for use with smaller or larger adults, children, or babies, without departing from the scope of this invention.

As depicted in FIGS. 2 and 5, the cover 22 and the compliant support 24 are adapted for sealing engagement with each other. The cover 22 is hinged to the support along an edge 60 of the support 24 to ensure that the cover and support remain attached and properly aligned for use with respect to one another. As illustrated, the cover 22 includes two first sealing portions 62 and the support 24 includes two second sealing portions 64 for engaging with the first sealing portions 62. One pair of sealing portions (i.e., one first sealing portion 62 and one second sealing portion 64) extends longitudinally adjacent the right side of the enclosure 14, and the second pair of sealing portions 62, 64 extends longitudinally adjacent the left side of the enclosure 14. In another configuration (not shown), the sealing portions 62, 64 are joined along edge 60 thereby providing a continuous seal for allowing the cover 22 to be completely removed from compliant support 24. The sealing portions 62, 64 comprise slide fastener members, such as the FLEXIGRIP 7 manufactured by MiniGrip/ZIP-PAK, an ITW Company, of Orangeburg, N.Y., USA, which are selectably sealingly engageable with one another. In another configuration (not shown), the sealing portions 62, 64 comprise a hook and loop fastening system. For example, a strip of hook material may be adhered to the compliant support 24, and a strip of loop material adhered to the cover 22 for engaging the hook material located on the compliant support. It is understood that the loop material can be placed on the compliant support 24 and the hook material on the cover 22.

The cover 22 may be slightly smaller than the support 24 which allows the sealing portions 62, 64 of both the cover and the compliant support to lie above and laterally inward from the sides of the support. As a result, the sealing portions 62, 64 are positioned away from the medial line of the patient 12 received in the interior space 16 of the enclosure 14 thereby allowing CPR to be administered to the patient without interference from the sealing portions. Alternatively, the cover 22 may be larger than the support 24, in which case the oversized cover can drape more conformably over the contours of the body, more effectively trapping a layer of coolant against the skin for enhanced heat exchange. Furthermore, the sealing portions 62, 64 are positioned on a portion of the enclosure that is maintained generally horizontal. As a result, the potential for the sealing portions 62, 64 to be bent or otherwise deformed is minimized. Bending and deformation of the sealing portions 62, 64 may diminish the ability to seal or to be opened or closed. Moreover, the sealing portions 62, 64 are positioned at a location above the depth D at which heat transfer liquid 18 accumulated in the well 58 of the compliant support 24, which reduces the demand on the sealing portions (i.e., the sealing portions do not have to form water tight seals). Lastly, the sealing portions 62, 64 are conveniently located for a user thereby providing the user with easy access to the patient 12.

With reference to FIGS. 6 through 8, both the impermeable member 26 of the cover 22 and the impermeable member 48 of the compliant support 24 include a flexible sheet-like body-facing component 66 and a flexible sheet-like outer component 67 that are adapted for face-to-face engagement with one another. The body-facing and outer components 66, 67 are liquid impermeable and joined to one another along their facing sides to form at least one passage 68 therebetween for the heat transfer fluid (FIGS. 2 and 5). Heat sealing is used to seal the components together along a seam 69 to form the passage 68 because it provides adequate strength without requiring additional raw materials. Other methods of forming the passages 68 or sealing the components 66, 67 to one another, such as adhesives, are also contemplated as being within the scope of the present invention. The passages 68 have a length approximately equal to the length of the cover 22, a width of approximately 25 mm, and a height of approximately 3 mm. It is understood that the dimensions provided for the passages 68 are exemplary only and that the passages can be formed to have various dimensions.

The passages 68 are configured to distribute fluid over a large portion of the surface area of the patient's body. As shown in FIGS. 2 and 5, the passages 68 comprise three passages extending generally longitudinally of the enclosure in each the impermeable member 26 of the cover 22 and the impermeable member 48 of the compliant support 24. Accordingly, three of the six total passages 68 are disposed above the patient's body while the other three passages are disposed beneath the patient's body. At least two of the passages 68 are arranged to engage the patient's body at a position offset from the medial line of the patient's body. This feature is particularly useful where CPR is to be administered to the patient 12, because chest compressions occur generally along the medial line of the patient. Where the patient 12 is placed within the enclosure 14 and the passage 68 corresponds approximately with the medial line of the patient, chest compressions may repeatedly block the flow of heat transfer fluid through the passage, thereby reducing fluid flow through the enclosure 14. Where at least some of the passages 68 are offset from the medial line of the patient 12, chest compressions performed in rendering CPR treatment are less disruptive of fluid flow through the enclosure 14. Other configurations of the passages 68 are also contemplated as being within the scope of the present invention. It is understood that the cover 22 and compliant support 24 may have more or fewer passages 68 without departing from the scope of this invention. It is also understood that the cover 22 may have a different number of passages than the compliant support 24. For example, the cover 22 could have about seventeen passages 68 closely spaced together with each passage having a width of approximately 1.2 centimeters (0.5 inches). In this configuration, the compliant support 24 could also have seventeen passages 68, more than seventeen passages, or fewer than seventeen passages. The passages 68 in the compliant support 24 could also be wider or narrower than the passages in the cover 22.

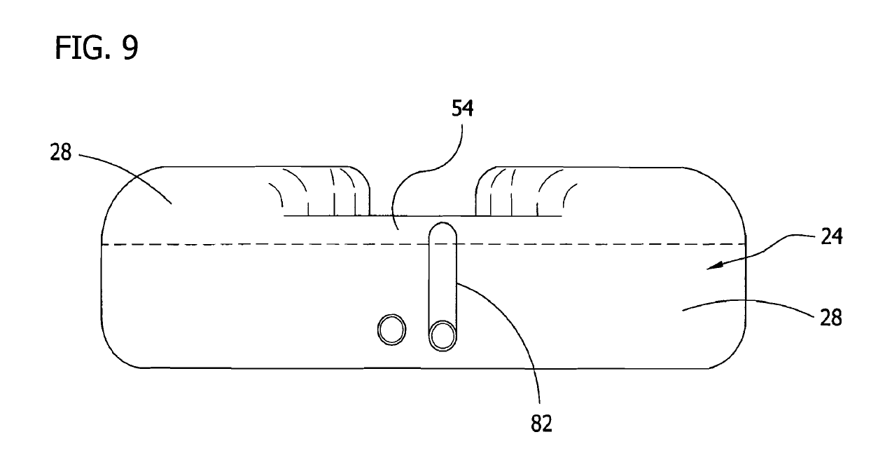

Referring now to FIG. 8, the passages 68 formed in the impermeable member 48 of the compliant support 24 are each supported by a hold-open 70, which holds the passage open and permits flow of the heat transfer fluid through the passage past the hold-open. The hold-opens 70 provide the rigidity necessary to maintain the passages 68 open even when subjected to a load, such as the weight of the patient's body which bears on the passages 68 formed in the impermeable member 48 of the support 24. The hold-open 70 may be a porous material, such as open-celled foams, particulate matter (e.g., polystyrene beads), batting, non-woven materials, or mechanical devices, such as coil springs. One suitable open-celled foam is a reticulated polyurethane foam having approximately 25 pores per inch manufactured by Foamex of Eddystown, Pa., USA, and sold under the trade name SIF.

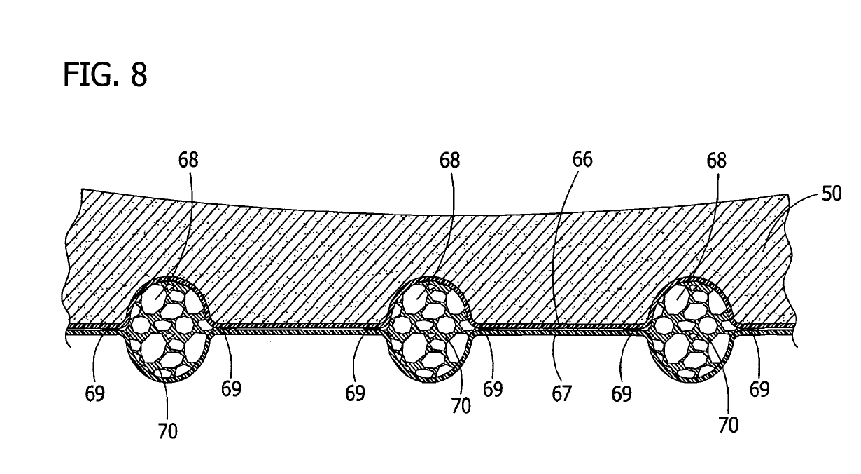

The passages 68 formed in the impermeable member 26 of the cover 22 are free of hold-opens 70 (FIG. 7). As a result, before the passage 68 fills with heat transfer fluid, the sheet-like body-facing component 66 and sheet-like outer component 67 of the passage generally lie flat against one another. Once heat transfer fluid flows inside the passage 68, the cross-sectional area of the passage increases to allow fluid to flow between the components. It is to be understood that the passages 68 formed in impermeable member 48 of the compliant support 24 may be substantially free of hold-opens 70 and the passages 68 formed in the cover 22 may have hold opens.

Referring again to FIGS. 5 and 6, the body-facing component 66 of both the cover 22 and the compliant support 24 have at least one opening 72 (i.e., an inlet) therein corresponding to the passage 68 for allowing the heat transfer fluid to pass from the passage to the porous layer 50 situated between the body-facing component 66 and the portion of the patient's body. Each inlet 72 is generally circular and preferably has a diameter of about 1 millimeter (0.04 inches). The small diameter inlets 72 restrict the flow of heat transfer fluid from the passage 68 into the enclosure 14 thereby causing the entire length of the passages to fill with heat transfer fluid. Thus, the heat transfer fluid is evenly distributed via the passage 68 to each of the inlets 72. The body-facing components 66 of the impermeable member 26 of the cover 22 and the impermeable member 48 of the compliant support 24 are disposed above and below the patient's body, respectively, thereby arranging the inlets 72 on opposite sides of the patient. As shown in FIG. 5, the body-facing component 66 of the impermeable member 48 of the support 24 has a plurality of inlets 72. The body-facing component 66 of the impermeable member 26 of the cover 22 also has a plurality of inlets (not shown) arranged in a similar manner to the inlets 72 of the compliant support 24.

The number of inlets 72 positioned in various portions of the enclosure 14 may be varied to regulate the distribution of heat transfer fluid throughout the enclosure. As illustrated in FIG. 5, the inlets 72 are positioned for evenly distributing the heat transfer fluid throughout the enclosure 14. However, it is understood that the inlets 72 may be positioned to distribute heat transfer fluid unevenly to the enclosure 14. By having an uneven flow distribution, a greater volume of heat transfer fluid can be directed to selected portions of the patient's body, such as those more amenable to heat transfer (i.e., the head, neck, torso) than other non-selected portions of the patient's body, which are also received in the enclosure 14.

As shown in FIG. 5, the passages 68 in the impermeable member 26 of the cover 22 and in the impermeable member 48 of the compliant support 24 extend through the rear end panel 54 located adjacent the bottom of the enclosure 14. As a result, heat transfer fluid directed through the passages 68 flows from a bottom section B (i.e., the lower one-third) of the enclosure 14, through a middle section M (i.e., the middle one-third) to a top section T (i.e., the top one-third). To even the flow distribution, the number of inlets 72 increases along the passage 68 in a direction away from the bottom section B of the enclosure. Thus, the middle section M has a greater number of inlets 72 than does the bottom section B, and the top section T has a greater number of inlets than does the middle section. In the illustrated configuration, each passage 68 is in fluid communication with four inlets 72 located in the bottom section B, six inlets located in the middle section M, and sixteen inlets located in the top section T. In another configuration (not shown), the diameters of the inlets 72 are varied along the passage 68 in a direction away from the bottom section B of the enclosure. Using this approach, inlets 72 having smaller diameters are positioned near the bottom sections B while inlets with progressively larger diameters are positioned in the middle and top sections M, T. It is understood that numerous inlet 72 configurations are possible to adequately distribute heat transfer fluid to the body of the patient 12 by varying the size, shape, and distribution of the inlets.

The enclosure 14 also comprises at least one large diameter (e.g., 2.5 centimeters (1 inch)) outlet 80 extending through the rear end panel 54 of the compliant support 24 for exhausting heat transfer fluid 18 from the enclosure 14 (FIG. 3). It is contemplated that the large diameter outlet 80 may be larger or smaller than 2.5 centimeters. The outlet 80 is sufficiently sized to allow heat transfer liquid 18 to be exhausted from the enclosure 14 by gravity at a rate equal to or greater than the rate at which the heat transfer liquid is being delivered to the interior space 16 of the enclosure 14 to thereby prevent the enclosure from overflowing. The enclosure 14 may have more than one outlet 80, the outlet may be positioned at other sections of the enclosure, and the outlet may have other sizes and shapes.

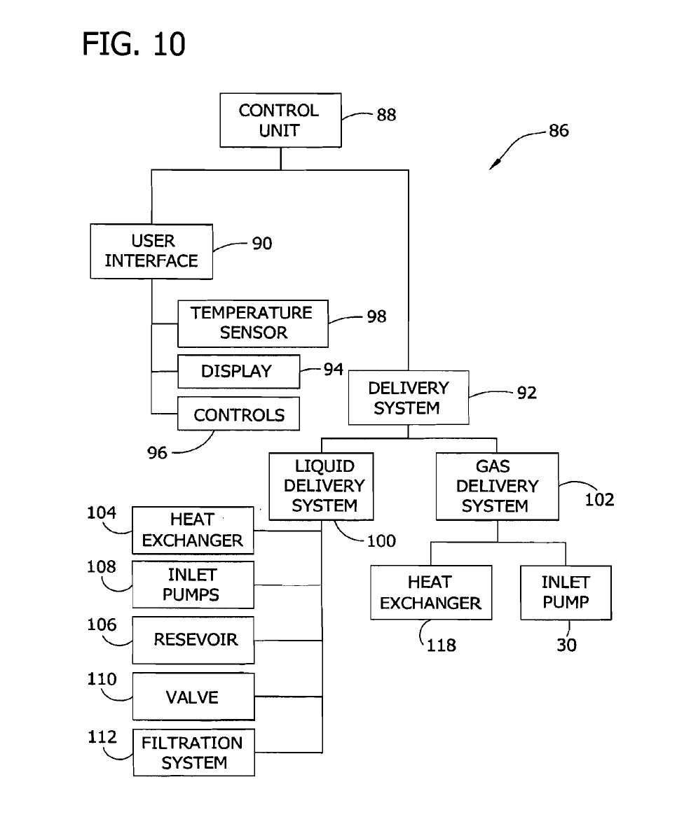

As shown in FIG. 3, an inverted U-shaped tube 82 (broadly, “a flow restrictor”) is in fluid communication with the outlet 80 for maintaining the depth D of the heat transfer liquid 18 within the enclosure 14 at a predetermined level thereby allowing the heat transfer liquid to accumulate in the well 58 of the compliant support 24 adjacent and beneath the patient 12. The inverted U-shaped tube 82 has a predetermined height thereby creating a spillway which the heat transfer fluid must flow over before it is exhausted from the enclosure (See FIG. 9). For instance, if the heat transfer liquid 18 is maintained at a depth of between about 7 centimeters (2.8 inches) and about 15 centimeters (6 inches) in the enclosure 14, the tube 82 needs to have a height sufficient to prevent transfer liquid below the selected height from flowing through the outlet 80 and out of the enclosure. Since the tube 82 maintains fluid at a given height at the outlet 80 of the enclosure 14, it creates a positive gage pressure at the outlet of the enclosure, which would between about 0.69 kilopascals (0.1 pounds per square inch) and about 1.47 kilopascals (0.2 pounds per square inch) for an enclosure with a depth of heat transfer liquid between 7 centimeters (2.8 inches) and about 15 centimeters (6 inches). A vent 84 is positioned on the tube 82 to provide an air break to thereby prevent siphoning of the heat transfer liquid 18 from the enclosure 14. The vent 84 can be selectively closed to facilitate siphoning, which may be advantageous when the heat transfer liquid 18 is being exhausted from the enclosure 14. It is contemplated that the tube 82 may be transparent to view the level of heat transfer liquid 18 contained in the enclosure 14. It is understood that the flow restrictor may be a device besides an inverted U-shaped tube 82, such as an adjustable valve, without departing from the scope of this invention.

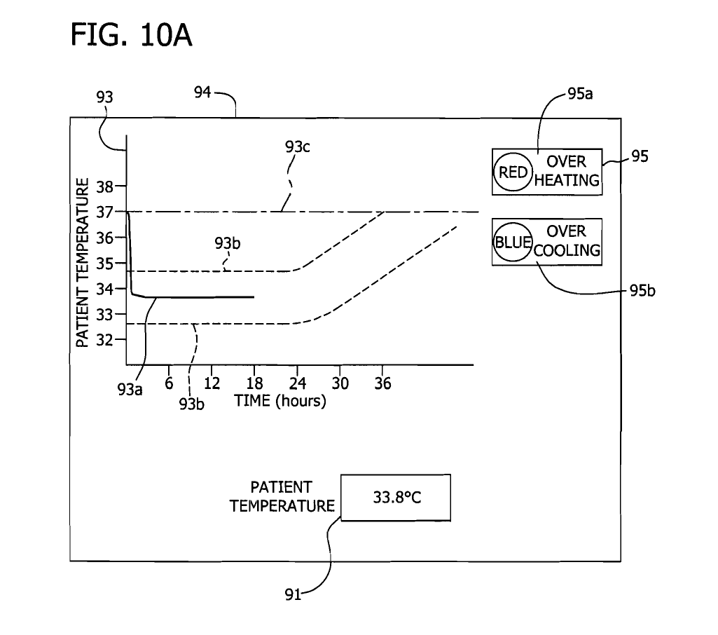

Referring now to FIG. 10, the apparatus further comprises a control system, generally indicated at 86, for controlling operation of the apparatus 10. The control system 86 includes a control unit 88 having a user interface 90, and a delivery system 92. The user interface 90 includes a display 94 for visually indicating particular parameters of the control system 86, controls 96 that allow the user of the system to selectively control particular system functions, and one or more temperature sensors 98 for measuring the temperature of the patient 12. For example, the controls 96 may allow the user to input a set-point, or target, body temperature for the patient 12. The display 94, for example, could display this set-point temperature along with the actual body temperature of the patient 12, the temperature of the heat transfer liquid 18, and the flow rate of the heat transfer liquid, among other things.

In one suitable example, the display 94 includes at least one source of information regarding the temperature of the patient 12. In the embodiment illustrated in FIG. 10A, for example, the display 94 is adapted to display information regarding the temperature of the patient 12 in three different ways. The first way is a directed readout 91 of the patient's body temperature. In the illustrated example, the displayed temperature is 33.8° C. It is understood that the temperature could be displayed in Fahrenheit in addition to or instead of Celsius. It is also understood that other conventional temperature scales could be displayed.

A second way comprises a graphical display 93 illustrating the patient's temperature versus time. A heavy line 93a of the graph illustrates the patient's temperature over time. As shown in the illustrated example, the patient 12 was rapidly cooled thereby altering the patient's temperature from about 37° C. to about 33.8° C. where it has been approximately maintained for about 18 hours. Dashed lines 93b are located above and below the line 93a representing the patient's body temperature and represent an upper limit (e.g., 34.5° C.) and a lower limit (e.g., 32.5° C.), respectively, for the patient's temperature. A linear dashed-dot 93c line represents the normal temperature of the patient (i.e., about 37° C.). It is understood that the temperature can be illustrated in any suitable temperature scale besides Celsius (e.g., Fahrenheit) and the time can be illustrated in any suitable time scale besides hours (e.g., seconds, minutes, days).

The third way comprises a visual warning display 95 that appears on the display 94. The illustrated warning display 95 includes two warning indicators 95a, 95b with each comprising both a color indicator (red, blue) and a text indicator (“Over Heating”, “Over Cooling”). If the patient's temperature reaches a temperature above the upper limit, one of the warning indicators 95a will be activated thereby displaying an “Over Heating” message and a red light. If the patient's temperature falls below the lower limit, the other warning indicators 95b will be activated thereby displaying an “Over Cooling” message and a blue light. It is understood that an audible warning indicator can be associated with each of the warning indicators 95a, 95b.

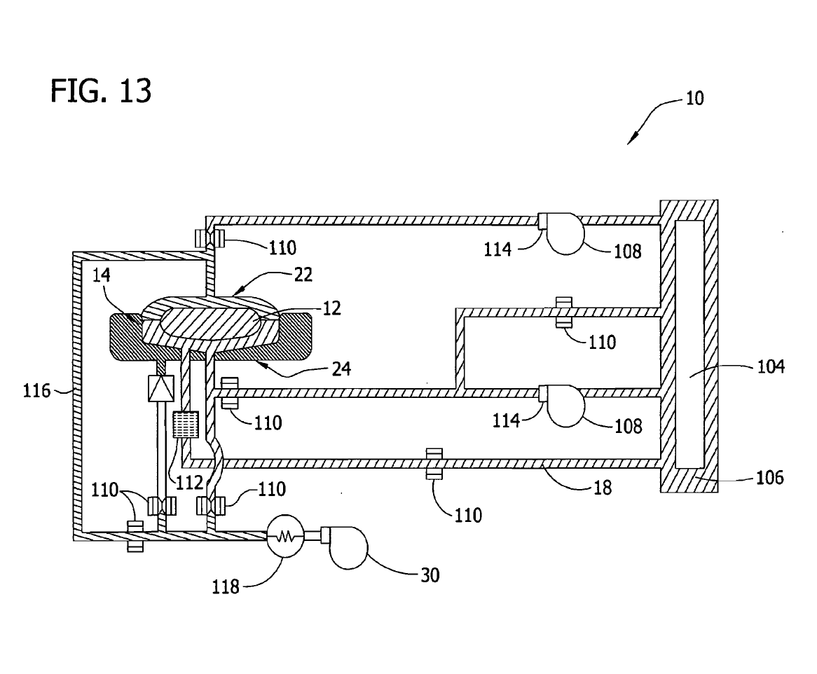

The delivery system 92 of the control system 86 comprises a liquid delivery system 100 which is a generally closed, continuous flow system in which heat transfer liquid 18 exhausted from the outlet 80 is directed to flow back to the passages 68 of the enclosure 14 for flow through the inlets 72 and into the interior space 16 of the enclosure (FIG. 12), and a gas delivery system 102 that delivers pressurized air to inflate the tubes 28 of the compliant support 24 (FIG. 11) and to flow into the enclosure 14 for direct contact with the patient's body (FIG. 14). With reference to FIGS. 12 and 13, the delivery system 92 comprises a liquid heat exchanger 104, a gas heat exchanger 118, a fluid reservoir 106, three pumps (two liquid pumps 108 and one air pump 30), a plurality of valves 110, and a filtration system 112. It is understood that the delivery system 92 can have fewer or more components without departing from the scope of this invention.

The heat exchanger 104 of the liquid delivery system 100 is used to alter the temperature of the heat transfer liquid 18 to an inlet temperature Ti, measured before the liquid enters the enclosure 14. Heat transfer liquid 18 exhausted from the enclosure 14 may be reintroduced into the enclosure as described above after passing through the heat exchanger 104. The heat exchanger 104 alters the temperature of the exhausted heat transfer liquid 18 from an outlet temperature To, measured after the liquid exits the enclosure 14, to the inlet temperature Ti. This allows the same heat transfer liquid 18 to be used repeatedly between the enclosure 14 and the liquid delivery system 100. Various types of heat exchangers 104 are contemplated as being within the scope of the present invention. For instance, the heat exchanger 104 of the present invention may incorporate a Peltier device and/or a phase-change material to facilitate returning the heat transfer liquid 18 to its inlet temperature Ti after passing through the enclosure 14 and being altered by the temperature of the patient's body. It is understood that the heat exchanger 104 can be used to warm or cool the heat transfer liquid 18. In the illustrated embodiment, the heat exchanger 104 is approximately 22 pounds of a phase change material (e.g., ice) placed in the reservoir 106 for direct contact with the heat transfer liquid 18 within the reservoir. It is appreciated that more or less of the phase change material may be used and that the heat exchanger 104 can be placed at other locations in the liquid distribution system 100.

The reservoir 106 holds heat transfer liquid 18 at the temperature induced by the heat exchanger 104 and stores it before the inlet pumps 108 pump the liquid into the enclosure 14. The reservoir 106 may have insulation (not shown) to help maintain the temperature of the heat transfer liquid 18 before it is pumped into the enclosure 14. Although various sized reservoirs may be used, the reservoir 106 in the illustrated embodiment has a capacity of about 40 liters (10.5 gallons). It is understood that reservoirs having different capacities may be used. For example, the reservoir 106 for holding heat transfer liquid 18 for the child or baby sized enclosure 14 may have a smaller capacity where as a reservoir for holding heat transfer liquid for a larger enclosure would have a larger capacity.

As shown in FIG. 12, two of the pumps are inlet pumps 108 in fluid communication with the reservoir 106 and the passages 68 of the enclosure 14 for pumping heat transfer liquid 18 from the reservoir into the enclosure at a flow rate of about 14 liters per minute (3.6 gallons per minute). As illustrated, one of the inlet pumps 108 directs heat transfer liquid to the passage 68 in the impermeable member 26 of the cover 22 for directing heat transfer liquid 18 over the top of the patient body, and the other inlet pump directs heat transfer liquid to the passage in the impermeable member 48 of the compliant support 24 thereby directing heat transfer liquid underneath the patient's body. Each of these pumps 108 can be operated independently of the other. Accordingly, heat transfer liquid 18 can be selectively directed for flow over the top of the patient's body, underneath the patient's body, or both (i.e., over the top of the patient's body and underneath the patient's body).

The pumps 108 may be a gear pump, such as utilized in the ThermoSuit® System manufactured by Life Recovery Systems, Waldwick, N.J., USA, or a roller-type pumphead with a motor drive, such as the 500 series process pump manufactured by Watson-Marlow OEM of Paramus, N.J., USA. Moreover, the pumps may have detachable pumpheads 114 such as the Pump Cassette Assembly manufactured by Life Recovery Systems, Waldwick, N.J. USA, that are disposable to minimize the likelihood of cross-contamination to subsequent patients. The pumpheads 114 are the only part of the pump 108 that contacts the heat transfer liquid 18. For example, the pumphead 114 may be made from a relatively inexpensive plastic material and easily attachable and detachable from the pump 108. For example, the pumpheads 114 may be made be from a plastic material and attached to the pump 108 using a pivoting hold-down bracket. Thus, after use, the pumphead 114 can be removed from the pump 108, discarded properly, and a new pumphead installed on the pump for use with another patient. Should higher flow rates or other parameters be required, alternative pumps, such as higher capacity gear or centrifugal pumps, may be used without departing from the scope of the present invention.

The filtration system 112 is in fluid communication with the outlet 80 of the enclosure 14 for filtering the heat transfer fluid 18 as it is exhausted thereby preventing potential contamination with other components of the liquid delivery system 100 (i.e., the inlet pumps 108 and reservoir 106). The filtration system 112 comprises a particular matter filter, activated carbon, and an ultraviolet light to kill bacteria and viruses. One such filtration system is the Aqua Sun Model SWP-V2 manufactured by Aqua Sun International, of Minden, Nev., USA. The filtration system 112 can be located anywhere within the liquid delivery system 100 or have more or fewer filtration capabilities without departing from the scope of this invention.

The air delivery system 102 comprises an air pump 30, such as a conventional reciprocating or scroll-type compressor, in fluid communication with the compliant support 24 for inflating the tubes 28 (FIG. 11), and the enclosure 14 for directing air 116 (broadly, “heat transfer gas”) into the enclosure (FIG. 14). Apart from its function of supplying a heat transfer gas 116, the air pump 30 is adapted to fill the tubes 28 of the compliant support 24 with air. For example, the pump 30 may have the capacity to fill the tubes 28 of the compliant support 24 with air at a rate of about 500 liters per minute to a positive gauge pressure of about 2 kilopascals (0.3 pounds per square inch). It is understood that other types of air pumps can be used and that the air pumps can have different flow rates then those indicated.

The air pump 30 is also used to pump air into the enclosure for heat transfer purposes (FIG. 14). The air heat exchanger 118, such as an inline air heater or cooler, can be used to alter the temperature of the air prior to it being pumped into the enclosure. Accordingly, the temperature altered air 116 can be directed into the enclosure to adjust the temperature of the patient received in the enclosure. This feature is particularly useful when heat transfer liquid 18 or phase change materials are unavailable. Moreover, temperature altered air 116 can be used to maintain the temperature of the patient 12 at a target temperature. For example, the heat transfer liquid 18 can be directed into the enclosure 14 to rapidly adjust the temperature of the patient 12 to, or near, a target temperature, and then temperature altered air 116 can be used to maintain the temperature of the patient at the selected target temperature. In addition, warm air can be used to suppress shivering sometimes experienced by patients whose temperature has been lowered.

As shown in FIGS. 11-14, the valves 110 provide control over the flow paths of both the heat transfer liquid 18 and the heat transfer gas 116 through the delivery system 92. The valves 110, such as pinch valves, are movable from a closed position in which the heat transfer liquid 18 (or heat transfer gas 116) is inhibited from flowing past the valve, to an open position where the heat transfer liquid (or heat transfer gas) is uninhibited to flow past the valve. For example, one of the valves 110 is positioned along the flow path between the passage 68 formed in the impermeable member 48 of the compliant support 24 and the reservoir 106. In the closed position, this valve 110 inhibits flow past the valve to the reservoir 106 and thereby allows the heat transfer liquid 18 to be pumped by the inlet pump 108 into the bottom of the interior space 16 of the enclosure 14. In the opened position and with the inlet pump 108 shut off, the valve 110 allows the heat transfer liquid 18 to flow via gravity through the passage 68 in the impermeable member 48 of the compliant support 24 and past the valve to the reservoir 106. The other valves 110 of the apparatus 10 control flow in other sections of the delivery system 92 in a similar manner. Other types of valves and other valves configurations are contemplated as being within the scope of this invention.

In operation, the enclosure 14 is placed on a generally flat surface, such an ambulance gurney 20. The compliant support 24 is fully extended to a position such that the underside of the compliant support is resting on the gurney. The cover 22 is disengaged from the compliant support 24, if necessary, and moved about the edge 60 toward the rear end panel 54 of the enclosure 14 thereby exposing the center of the compliant support 24. The patient 12 is carefully placed in the center of the compliant support 24 on the porous layer 50 overlying the impermeable member 48 and aligned with the positioner 56 (i.e., the face of the patient 12 is aligned with the image of a face) to ensure proper patient placement. The air pump 30 is then activated to inflate the tubes 28 to the desired pressure (FIG. 11), and thereby conform the interior surfaces 36 of the tubes 28 to the portion of the patient's body juxtaposed thereto. The air pump 30 can be activated anytime during use to maintain the tubes 28 at the desired pressure. The cover 22 is then positioned to cover the patient's body from the neck downward. The sealing portions 62, 64 of the cover 22 and the compliant support 24 are engaged thereby enclosing the patient 12 in the enclosure 14.

Using the control unit 88, the delivery system 92 is then activated to deliver either heat transfer liquid 18 or heat transfer gas 116 to the patient's body to adjust the temperature of the patient 12 to a selected temperature (FIG. 12). For example, it may be desirable to quickly lower the body temperature of a patient 12 suffering from cardiac arrest from about 37° C. (98.6° F.) to about 33° C. (91.4° F.). In this example, approximately 30 liters (7.9 gallons) of the heat transfer liquid 18 (e.g., water) and approximately 10 kilograms (22 pounds) of phase change material (e.g., ice) are added to the reservoir 106. It may be desirable to use pre-cooled heat transfer liquid 18. The heat transfer liquid 18, which is lowered to a temperature between about 0.5° C. (32.9° F.) and about 4° C. (39.2° F.) is then pumped through the passages 68 and inlets 72 and into the top and bottom of the enclosure 14 by the two inlet pumps 108 such that heat transfer liquid 18 is in direct contact with the patient's body at a flow rate of about 14 liters per minute (3.6 gallons per minute). The heat transfer liquid 18 below the patient's body flows through the passage created by the porous layer 50. In addition to being able to pump heat transfer liquid 18 into both the top and bottom of the enclosure 14 simultaneously, the inlet pumps 108 can be selectively operated to pump heat transfer liquid 18 only into the top of the enclosure or only into the bottom of the enclosure.

Heat transfer liquid 18 accumulates in the well 58 created by the patient 12 in the compliant support 24 such that a greater volume of heat transfer liquid accumulates in the region of the compliant support that receives the torso than the regions of the compliant support that receive the head, legs, and feet. The heat transfer liquid accumulates in the interior space 16 of the enclosure 14 until it reaches a height greater than the spillway created by the drain tube 82 in fluid communication with the outlet 80. The drain tube 82 maintains the heat transfer liquid 18 at a target depth of about 11 centimeters (4.5 inches), which creates a positive gauge pressure at the outlet 80 of the enclosure 14 of about 1.1 kilopascals (0.16 psi). Any heat transfer liquid 18 achieving a height greater than the spillway created by the drain tube 82 will be exhausted from the enclosure at a flow rate equal to or greater than flow rates at which the heat transfer liquid is being driven into the interior space 16 of the enclosure 14 by the inlet pumps 108.

The heat transfer liquid 18 exhausted from the enclosure 14 passes through the filtration system 112 to remove contamination from the patient 12, such as particulate matter, viruses, and bacteria. The filtered heat transfer fluid 18 is directed back into the reservoir 106 where it is re-cooled by the phase change material prior to being recirculated into the interior space 16 of the enclosure 14. Heat transfer fluid 18 is continuously recirculated through the enclosure 14 until the patient's temperature reaches or approaches the selected temperature. The patient's temperature may drop slightly even after the heat transfer liquid 18 has been stopped and, as a result, it may be desirable to stop the flow of heat transfer liquid short of the selected temperature to prevent overshoot (i.e., lowering the patient's body temperature below the selected temperature). At this point, the inlet pumps 108 are shut off and the heat transfer liquid 18 is exhausted from the enclosure 14 via gravity. Once the inlet pumps 108 are shut off, the valves 110 are adjusted to allow heat transfer liquid 18 to be exhausted from the interior space 16 of the enclosure 14 though the inlets 72 in communication with the passages 68 in the compliant support 24 (FIG. 13). The air pump 30 can be used to pump air into the top of the enclosure 14 to more rapidly exhaust the heat transfer liquid 18 from the enclosure. Further yet, the inlet pump 108 in fluid communication with the bottom of the enclosure 14 can be used as an outlet pump to pump heat transfer liquid 18 from the interior space 16 of the enclosure back to the reservoir 106.

The patient 12 can be maintained at the selected temperature by turning on the air pump 30 and directing the air pumped by the air pump through the inline heat exchange 118 (FIG. 14). The cooled heat transfer gas 116 is directed to flow into the passages 68 and through the inlets 72 of the enclosure 14 for direct contact with the patient's body. It is believed that the patient's body temperature can be maintained using heat transfer gas 116 for a desired period of time of 12 to 24 hours or more. The heat transfer gas 116 exits the interior space 16 of the enclosure 14 through the unsealed portion of the enclosure adjacent the patient's neck and/or the outlet 80.

The heat transfer gas 116 can also be used to induce slower temperature changes in the patient 12 than the heat transfer liquid 18 or to suppress shivering. In addition, heat transfer gas 116 can be used at remote locations away from the ambulance or a reservoir 106 of heat transfer liquid 18. This relieves the user of the need to transport heavy heat transfer liquid 18 and phase change materials to the patient without delaying treatment of the patient. After the patient has been transported to a suitable location (e.g., ambulance, hospital), heat transfer liquid 18 can be introduced into the interior space 16 of the enclosure 14.

It is understood that during the above mention operations, the user is able to maintain visual observation of the patient's body through the transparent cover 22. If additional medical care is needed, the cover 22 can be pulled back about edge 60 (or completely removed), with the delivery system operating 92, to expose the patient's body. The delivery system 92 will continue to direct the heat transfer liquid 18 or heat transfer gas 116 to the underside of the patient's body. If the liquid delivery system 100 is being used, the inlet pump 108 directing heat transfer liquid 18 to the passage 68 in the cover 22 can be shut off before the cover is pulled back to prevent any heat transfer liquid 18 from spilling from the apparatus 10. Moreover, all of the apparatus' operations can occur in the ambulance on route to the medical facility thereby not delaying any subsequent medical care.

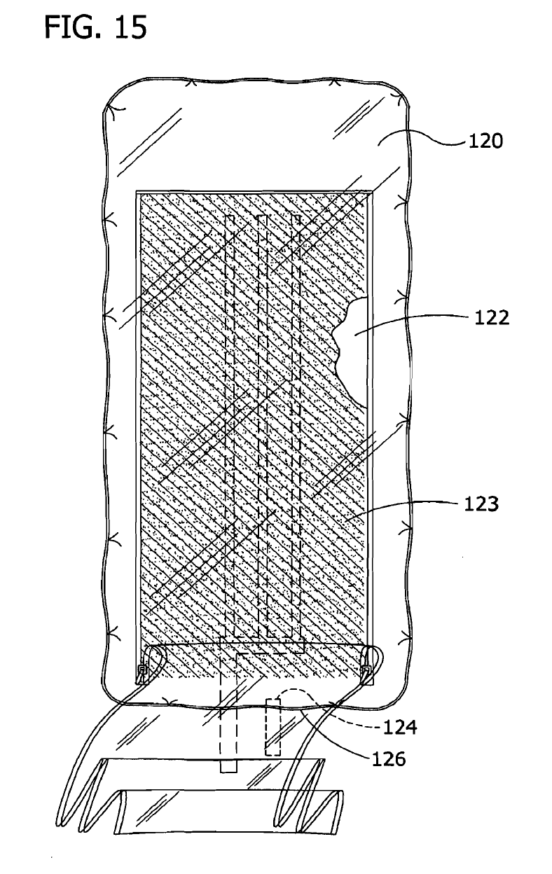

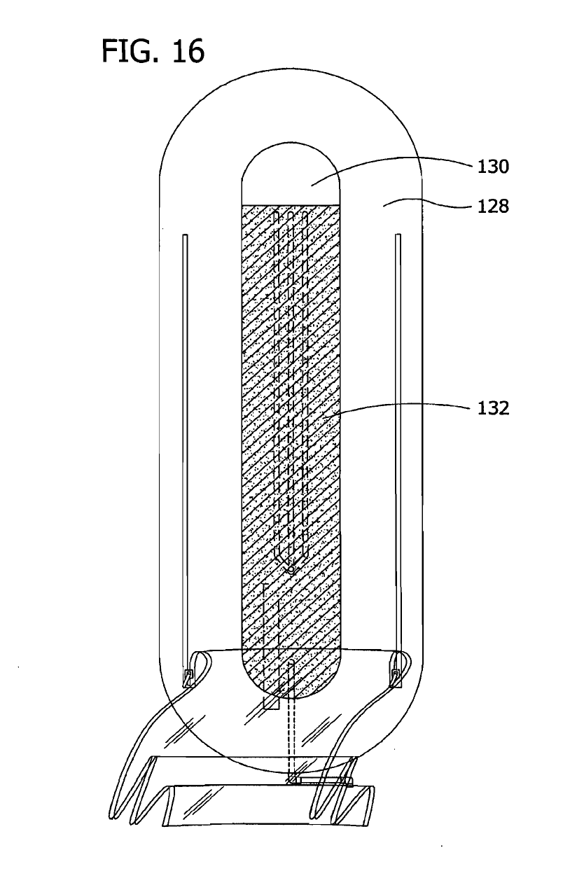

FIGS. 15 and 16 illustrate other configurations of the compliant support 24. The compliant support 24 illustrated in FIG. 15 comprises a generally rectangular air mattress 120. The air mattress 120 is only partially inflated thereby allowing a well to form under the weight of the patient (not shown) placed on the mattress. A rectangular impermeable member 122 of substantially the same construction as described above overlies and is affixed to the mattress 120. A batting layer 123 is placed over the impermeable member 122. A portion of the batting layer 123 is cut away in FIG. 15 to show the underlying impermeable member 122. An outlet 124 is fluid communication with the well 121 and a conduit extending through a rear end panel 126 of the mattress 120 for exhausting heat transfer liquid from the interior space of the enclosure.

In the configuration of FIG. 16, the compliant support 24 comprises an inflatable, oblong tube 128 extending around the entire periphery of the support. An impermeable member 130 of substantially the same construction as described above is located in the center of the oblong tube 128 and is bonded to an underside of the oblong tube about its entire circumference to define a water tight well 132 for receiving the patient's body.

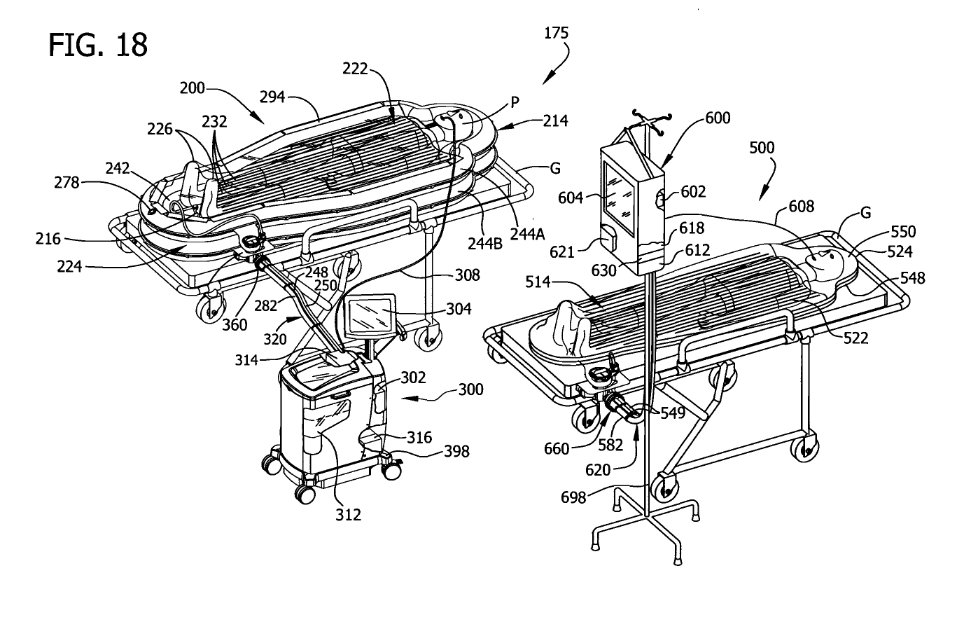

FIG. 18 illustrates a system, indicated generally at 175, for altering and maintaining the body temperature of a patient P. The system 175 comprises a first, liquid body temperature cooling/heating apparatus (broadly, a temperature reducing apparatus), generally indicated by reference number 200, for rapidly adjusting the body temperature of a patient P to approximately a target temperature and a second, gas body temperature cooling/heating apparatus (broadly, a temperature maintenance apparatus or a temperature maintenance and re-warming apparatus), generally indicated by reference number 500, for maintaining the body temperature of the patient at the target temperature of an extended period of time. As used herein “rapidly adjusting the body temperature of a patient” means to adjust the body temperature of the patient P to approximately the target temperature in less than one hour, suitably less than thirty minutes, and more suitably less than fifteen minutes. An “extended period of time” is used herein to mean a period of time exceeding four hours, suitably exceeding eight hours, and more suitably exceeding twelve hours.

The liquid body temperature cooling/heating apparatus 200 illustrated in FIG. 18 is described in some detail below but is also described in detail in U.S. patent application Ser. No. 11/466,966 filed on Aug. 24, 2006 and published on Dec. 14, 2006 as U.S. Patent Application Publication No. 2006/0282142. The '966 Application is incorporated herein by reference in its entirety. In one suitable embodiment, the liquid body temperature cooling/heating apparatus 200 comprises a ThermoSuit™ system available from Life Recovery Systems HD, LLC of Waldwick, N.J.

As seen in FIG. 18, the liquid cooling/heating apparatus 200 generally comprises an enclosure, indicated at 214, (broadly, “a first enclosure”) defining an interior space 216 for receiving at least a portion of the patient's body. The enclosure 214 is adapted to allow heat transfer liquid, such as water, saline, or other suitable liquids, to flow into the interior space 216 and into direct contact with the patient's body to promote heat transfer between the patient P and the heat transfer liquid. In the illustrated embodiment, the interior space 216 of the enclosure 214 is configured to receive substantially the entire body of the patient P, including the torso, arms, and legs. As a result, the amount of surface area of the patient P available for contact by the heat transfer liquid is maximized. It is to be understood that the enclosure 214 can be configured to receive less than the patient's entire body. That is, the enclosure 214 can be configured to receive only a portion of the patient's body. The illustrated enclosure 214 is adapted to generally conform to the shape of the body of the patient P received therein to accommodate patients of various shapes and sizes.

As illustrated in FIG. 18, the enclosure 214 comprises a cover, indicated at 222, for overlying the patient P from the neck downward, and a compliant support, indicated at 224, for underlying the patient's entire body. The cover 222 is limp so that it generally conforms, under its own weight, to the contours of the upward facing surface of the patient's body it is covering. To this end, the cover 222 includes two foot gussets or slits 226 located in a portion of the cover adapted to receive the feet of the patient P.

The cover 222 is liquid impermeable and includes a plurality of passages 232 for allowing the heat transfer liquid to flow through the cover. In the illustrated embodiment, the passages 232 are configured to distribute heat transfer liquid over a large portion of the surface area of the patient's body (i.e., from the neck downward). The weight of the heat transfer liquid flowing through the passages 232 causes the cover 222 to further conform to the contours of the patient's body. Since the passages 232 extend throughout much of the cover 222, the majority of the cover is weighted against the body of the patient P by the heat transfer liquid.

A plurality of openings or inlets (not shown) are formed in the cover 222 and in fluid communication with at least one of the passages 232 for allowing the heat transfer liquid to pass from the passages to the portion of the patient's body received in the enclosure. The number of openings positioned in various portions of the cover 222 may be varied to regulate the distribution of heat transfer liquid throughout the enclosure 214. It is understood that numerous configurations for the openings are possible to adequately distribute heat transfer liquid to the body of the patient P by varying the size, shape, and distribution of the openings. It is also understood that the openings in the cover 222 may be positioned to distribute heat transfer liquid unevenly throughout the interior space 216 of the enclosure 214. By having an uneven flow distribution, a greater volume of heat transfer liquid can be directed to selected portions of the patient's body, such as those more amenable to heat transfer (e.g., the head, neck, torso), than other non-selected portions of the patient's body, which are also received in the enclosure 214.

In the illustrated embodiment, the cover 222 is made of a transparent material, such as polyvinyl chloride (PVC), polyethylene, or polyurethane, so that the body of the patient P received within the interior space 216 of the enclosure 214 can be viewed through the cover. It is to be understood, however, that the cover 222 can be made of a non-transparent material or have a portion that is transparent and a portion that is non-transparent.

The compliant support 224 is a pneumatic support, which (like the cover 222) generally conforms to the shape of the patient's body when the body rests on the support. Moreover, the compliant support 224 minimizes pressure concentrations beneath the patient P which facilitates the flow of heat transfer liquid beneath the patient and minimizes the possibility of pressure sores developing in the skin of the patient. Generally, the compliant support 224 comprises an inflatable base 242 (broadly, a “first zone”), which is the portion of the compliant support upon which the patient P rests, and two generally oblong, inflatable tubes 244A, 244B (broadly, a “second zone”) forming a periphery around the base. In the illustrated embodiment, one of the inflatable tubes 244A is arranged on top of the other tube 244B. It is to be understood, however, that more or fewer (i.e., one) inflatable tubes 244A, 244B can be used to form the periphery of the base 242. It is also to be understood that the inflatable tubes could be disposed side-by-side instead of one on top of the other.

As seen in FIG. 18, the stacked inflatable tubes 244A, 244B and base 242 cooperatively form a watertight well for receiving the entire body of the patient P therein. The well is configured to generally conform to the body of the patient P thereby minimizing the volume of the interior space 216 of the enclosure 214 and the amount of heat transfer liquid necessary to effectively alter the body temperature of the patient P. More specifically, the patient P is positioned in a supine position on the base 242 with the base and the tubes 244A, 244B in a deflated state. The base 242 and inflatable tubes 244A, 244B are then inflated to enclose the patient's body within the well and generally conform the well to the profile of the patient's body. As the inflatable tubes 244A, 244B are filled with air (or other suitable gas), the tubes generally conform to the sides of the patient P. The base 242 is typically inflated to a pressure that is less than the inflated pressure of the inflatable tubes 244A, 244B. As a result, the base 242 easily conforms to the contours of the patient P because of the patient's weight. More specifically, the weight of the patient P causes the base 242 to assume a bowl-shape that is tailored to the patient's body. The base 242 and inflatable tubes 244A, 244B can be inflated manually or with an air pump. It is to be understood that the compliant support 224 may have different shapes and sizes or be conformable with the patient's body in a way different from that described herein.

A porous layer (not shown) is used to cover the well of the compliant support 224 so that the porous layer is disposed between the well and the body of the patient P. The porous layer, such as rich loft polyester batting or open-cell polyurethane foam, allows heat transfer liquid to flow between the body of the patient P and the well and thereby across the skin of the patient. The porous layer prevents areas of the well from being sealed off from the body of the patient P contacting the base 242, which would inhibit flow of heat transfer liquid beneath the body of the patient.

The base 242 includes a plurality of supply passages for allowing heat transfer liquid to be supplied beneath the body of the patient P, and at least one return passage for allowing heat transfer liquid to be drained from the compliant support 224. A plurality of openings or inlets is in fluid communication with the supply passages in the base 242 for allowing the heat transfer liquid to pass from the passage into direct fluid contact with the underside of the patient's body received in the enclosure 214, and a plurality of apertures or outlets is in fluid communication with the return passages for allowing heat transfer liquid to exit the enclosure 214. The return passages in the compliant support 224 are fluidly connected to at least one drain tube 282 for transferring heat transfer liquid away from the interior space 216 of the enclosure 214.

Each of the passages formed in the compliant support 224 are supported by a hold-open, which holds the passages open and permits flow of the heat transfer liquid through the passage past the hold-open. In other words, the hold-opens provide the rigidity necessary to maintain the passages open even when subjected to a load, such as the weight of the body of the patient P which bears on the passages formed in the well. The hold-open may be a porous material, such as open-celled foams, particulate matter (e.g., polystyrene beads), batting, non-woven materials, or mechanical devices, such as coil springs.

As seen in FIG. 18, the cover 222 and the compliant support 224 include sealing portions 294 adapted for selective engagement with each other. In the illustrated embodiment, the sealing portions 294 comprise a hook and loop fastening system. For example, a strip of hook material can be adhered to the compliant support 224, and a strip of loop material can be adhered to the cover 222 for engaging the hook material located on the compliant support. It is to be understood that the loop material can be placed on the compliant support 224 and the hook material on the cover 222. It is also understood that other types of fastening systems (e.g., adhesives, slide fasteners, snaps) can be used. It is further understood that a portion of the cover 222 can be bonded to the compliant support 224 to thereby hingedly attach the cover to the compliant support.

The liquid cooling/heating apparatus 200 further comprises a control system, generally indicated at 300, for controlling operation of the apparatus. The control system 300, which in the illustrated embodiment is mounted on a mobile cart 398, includes a controller 302, a monitor 304 (broadly, a “user interface”), a delivery system, and a temperature sensor 308 for measuring the temperature of the patient P. The monitor 304 includes an LCD touch screen display for visually indicating particular parameters of the control system 300 and for allowing the user of the system to selectively control particular system functions. The monitor 304, for example, could display a target temperature along with the actual body temperature of the patient P, and the temperature of the heat transfer liquid, among other things. With respect to user control of the system 300, the user can start, pause, and stop the delivery system using the touch screen display of the monitor 304. It is also understood that other system 300 functions could be controlled by the user using the touch screen display of the monitor 304.

The delivery system of the control system 300 comprises a liquid delivery system and an inflating system. The liquid delivery system is a generally closed, continuous flow system in which heat transfer liquid is cycled through the interior space 216 of the enclosure 214. The liquid delivery system comprises a fluid reservoir 312, at least one liquid inlet pump 314, and an umbilicus indicated generally at 320. The umbilicus 320 fluidly connects the reservoir 312 and the liquid inlet pump 314 to the interior space 216 of the enclosure 214. It is to be understood that the delivery system can have fewer or more components. Along with the heat transfer liquid, a phase change material (e.g., ice) can be placed into the reservoir 312 to alter and/or maintain the temperature of the heat transfer liquid to an inlet temperature, measured before the liquid enters the enclosure 214. Besides phase change materials, various other types of heat exchangers (e.g., Peltier device) are contemplated as being within the scope of the present invention.

The liquid inlet pump 314 is in fluid communication with the reservoir 312, the umbilicus 320, and the inlet passages in the enclosure 214 so that the pumps can pump heat transfer liquid from the reservoir into the enclosure. More specifically, the liquid inlet pump 314 directs heat transfer liquid into the passages in the cover 222 for directing heat transfer liquid over the top of the body of the patient P, and into the passages in the compliant support 224 thereby directing heat transfer liquid underneath the patient's body. It is understood that the liquid delivery system can include more than one liquid inlet pump 314 so that heat transfer liquid can be selectively directed for flow over the top of the body of the patient P, underneath the patient's body, or both (i.e., simultaneously over the top of the patient's body and underneath the patient's body).

The control system 300 further includes the inflating system for delivering pressurized air to inflate the various inflatable components of the compliant support 224. The inflating system comprises an air pump 316 and a plurality of pressure sensors. The air pump 316, such as a conventional reciprocating or scroll-type compressor, is in fluid communication with the compliant support 224 for inflating the base 242 and inflatable tubes 244A, 244B. In one configuration, the pump 316 may have the capacity to fill the inflatable tubes 244A, 244B of the compliant support 224 with air at a rate of about 500 liters per minute to a positive gauge pressure of about 3.4 kilopascals (0.5 pounds per square inch) and the base 242 to a positive gauge pressure of about 0.76 kilopascals (0.11 pounds per square inch). It is to be understood that other types of air pumps can be used and that the air pumps can have different flow rates then those indicated.

The pressure sensors are adapted to measure the air pressure within the inflatable tubes 244A, 244B and the base 242 of the compliant support 224 and are connected to the controller 302 so that their air pressure measurements are conveyed to the controller. The controller 302 is programmed to compare the detected pressure measurements to predetermined pressures and if the detected measurements differ from the predetermined pressures, the controller can activate the air pump 316 to bring the air pressures within the inflatable tubes 244A, 244B and the base 242 to about the predetermined pressures. Accordingly, should air leaks occur during operation of the apparatus 200, the air pump 316 will be activated, as necessary, to maintain the proper air pressures within the complaint support 224.

The umbilicus 320 is used to simply and easily connect the liquid inlet pump 314 and the air pump 316 to the enclosure 216. The umbilicus 320 includes two flexible air supply conduits 248 (only one being shown in FIG. 18) for supplying air from the air pump 316 to the inflatable tubes 244A, 244B and the base 242. Specifically, one of the air supply conduits 248 feeds the inflatable tubes 244A, 244B and the other air supply conduit feeds the base 242. The umbilicus 320 also includes at least one flexible liquid supply conduit 250 to fluidly connect the heat transfer liquid inlet pump 314 to the enclosure 216. The liquid supply conduit 250 is used to feed heat transfer liquid to the cover 222 and the compliant support 224. The umbilicus 320 further includes the drain tube 282 that feeds heat transfer liquid from the enclosure 214 back into the reservoir 312. Each end of the umbilicus 320 comprises a quick-connect coupling 360 (one being shown in FIG. 18) to attach the ends of the umbilicus to the control system 300 and the enclosure 216 to establish a fluid connect therebetween.

The apparatus 200 shown in the attached drawings is intended to be used in a medical treatment facility (e.g., a hospital). The enclosure 214, for example, is sized and shaped for placement on a stretcher, such as an ambulance or emergency gurney G, to facilitate the transportation of the patient P in a conventional manner while placed in the enclosure.

As mentioned above, the enclosure 214 is adapted to allow heat transfer liquid to flow into the interior space 216 for direct contact with the patient's body to promote heat transfer between the patient P and the heat transfer liquid. To raise the temperature of a patient P, the heat transfer liquid is directed into the interior space 216 of the enclosure 214 at a temperature greater than the temperature of the portion of the patient's body (broadly, a liquid warming mode of the system). For example, the heat transfer liquid may have a temperature in a range of about 37° C. (98.6° F.) to about 47° C. (117° F.), such as about 45° C. (113° F.). One application of such warming would be to warm a patient P suffering from unintended hypothermia.

To rapidly lower the temperature of a patient P, the heat transfer liquid is directed into the interior space 216 of the enclosure 214 at a temperature significantly lower than the temperature of the body portion of the patient (the normal core body temperature of a human patient is about 37° C. (98.6° F.)) received in the interior space 216 of the enclosure so that the fluid cools the body portion of the patient (broadly, a liquid cooling mode of the system). For example, the heat transfer liquid may have a temperature in a range of about 0° C. (32° F.) to about 5° C. (41° F.). Heat transfer liquid introduced into the enclosure 214 at such a temperature has been found to cool the body at a sufficient rate to induce hypothermia while minimizing any adverse effects to the skin of the patient P. It is to be understood that temperatures other than those listed above can be used to adjust the temperature of a patient P received in the interior space 216 of the enclosure 214.

One application of cooling would be to cool a patient P suffering from cardiac arrest. It is well recognized that organ damage can, and typically does, occur shortly after the victim has suffered cardiac arrest. As a result, it is often in the victim's best interest to quickly and effectively induce hypothermia to minimize or prevent organ damage. It is also contemplated that the apparatus 200 may be used to treat other medical conditions than those listed or have application in other medical procedures (e.g., hyperthermia, trauma, stroke, enhancements of anti-cancer therapies, surgical support, spinal injury, and general thermal management).

With reference to FIGS. 18-20, the second, gas body temperature cooling/heating apparatus 500 of the system 175 comprises an enclosure, indicated at 514, (i.e., a second enclosure separate from the first enclosure 214 of the liquid body temperature cooling/heating apparatus 200) defining an interior space 516 for receiving at least a portion of a patient's body. The enclosure 514 is also adapted to allow heat transfer gas, such as air or other suitable gases to flow into the interior space 516 for direct contact with the patient's body to promote heat transfer between the patient P and the heat transfer gas. While it is understood that any portion of the patient's body (including the entire body) may be placed inside the enclosure 514, for exemplary purposes, the illustrated portion of the patient's body received in the interior space 516 of the enclosure 514 is the patient's body from the neck downward, including the torso, arms, and legs. The enclosure 514 is adapted to accommodate patients of various shapes and sizes. For example, in one configuration, the enclosure 514 is suitable for individuals having a size between about the 5th percentile and about the 95th percentile adult male. Enclosures adapted to receive smaller individuals (e.g., babies, children, small adults) or larger individuals are also contemplated.

The illustrated enclosure 514 is sized and shaped for placement on a stretcher, such as an ambulance or emergency gurney G, with the patient P received in the interior space 516 of the enclosure. Accordingly, the enclosure 514 may have a width between about 66 centimeters (26 inches) and about 76 centimeters (30 inches) and a length between about 203 centimeters (80 inches) and about 210 centimeters (83 inches), the approximate range of dimensions for a standard ambulance or emergency gurney G. It is contemplated that the enclosure 514 may have other configurations without departing from the scope of this invention.

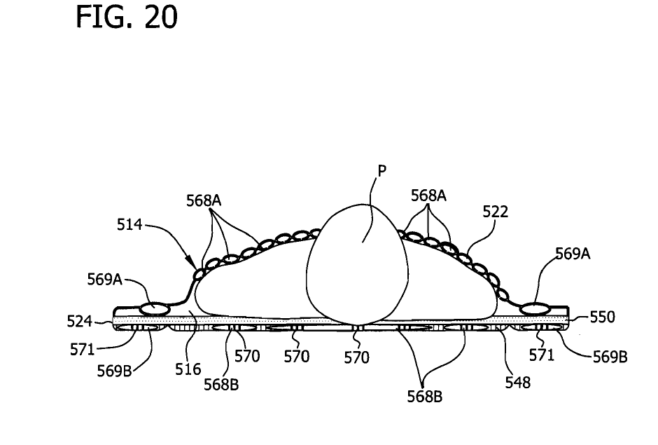

In the illustrated configuration, the enclosure 514 is adapted to enclose the patient's body from the neck down thereby providing a large portion of the patient's total surface area for heat transfer with the heat transfer gas. As illustrated in FIG. 18, the enclosure 514 comprises a blanket 522 for overlying the patient P from the neck downward, and a base 524 for underlying the patient's entire body. As seen in FIGS. 18 and 20, the blanket 522 comprises a limp sheet-like member adapted to generally conform, under its own weight, to the contours of the patient P which it is covering. The sheet-like member is preferably made of a transparent material such as polyvinyl chloride (PVC), polyethylene, polyethylene terephthalate (PET) or polyurethane so that the body of the patient received within the enclosure can be viewed. It is understood, however, that a sheet-like member (not shown) may be made of a non-transparent material or has a portion that is transparent with the remainder of sheet-like member being non-transparent.

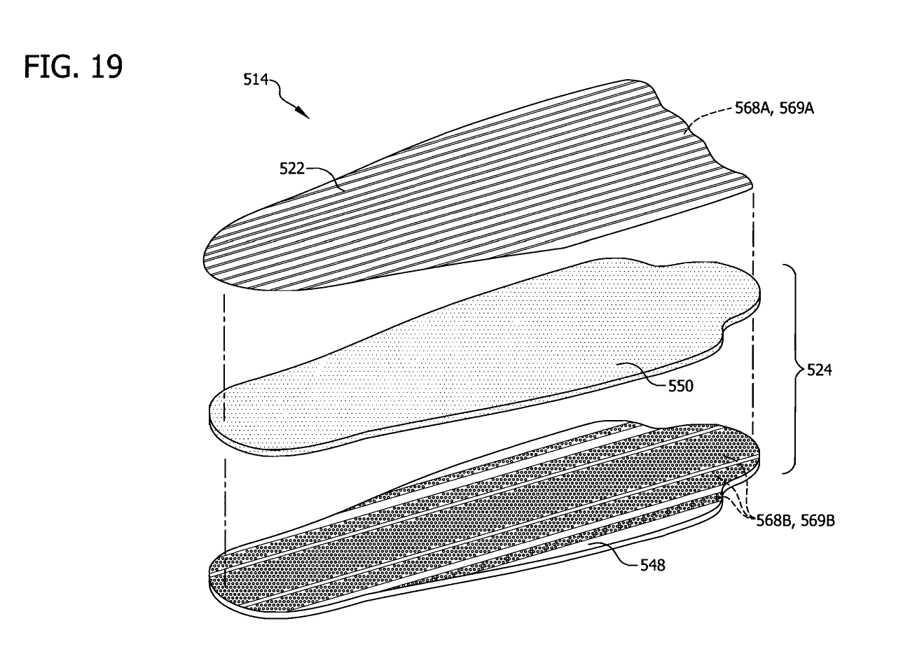

Referring to FIG. 19, the base 524 comprises a bottom, vapor impermeable sheet-like member 548 and a porous layer 550 overlying the member. The vapor impermeable member 548 retains the heat transfer gas within the enclosure 514. The vapor impermeable member 548 comprises a transparent material such as PVC, polyethylene, PET, or polyurethane. The porous layer 550, which can be, e.g., a rich loft polyester batting or an open-cell polyurethane foam, allows heat transfer gas to pass into contact with the patient's body portion for flow across the skin throughout the enclosure 514. In one suitable embodiment of the gas heating/cooling apparatus 500, the base 524 is omitted. In this embodiment, the patient P can lie directly on the gurney G or any suitable supporting surface (e.g., a hospital bed) and be covered by the blanket 522.

In one embodiment and as illustrated in FIG. 20, the blanket 522 and the base 524 are adapted for sealing engagement with each other. As illustrated, an adhesive can be used to engage the blanket 522 and the base 524. In another configuration, the blanket 522 and cover can be sealing engaged using a hook and loop fastening system. For example, a strip of hook material may be adhered to the base 524, and a strip of loop material adhered to the blanket 522 for engaging the hook material located on the base. It is understood that the loop material can be placed on the base 524 and the hook material on the blanket 522. In yet another configuration, the blanket 522 can be weighted about its peripheral edge for engaging the blanket 522 and the base 524. It is understood that any suitable engagement system can be used. It is also understood that the blanket 522 can overlie the base 524 in a non-sealing engaged manner.

Both the blanket 522 and the base 524 include a plurality of inlet passages 568A, 568B configured to distribute gas over a large portion of the surface area of the patient's body. The passages 568B formed in the base 524 are each supported by a hold-open 570, which holds the passage open and permits flow of the heat transfer gas through the passage past the hold-open. The hold-opens 570 provide the rigidity necessary to maintain the passages 568B open even when subjected to a load, such as the weight of the patient's body which bears on the passages formed in the base 524. The hold-open 570 may be a porous material, such as open-celled foams, particulate matter (e.g., polystyrene beads), batting, non-woven materials, or mechanical devices, such as coil springs. In the illustrated embodiment, the passages 568A formed in the blanket 522 are free of hold-opens 570 (FIG. 20). It is to be understood, however, that the passages 568B formed in base 524 may be substantially free of hold-opens 570 and the passages 568A formed in the blanket 522 may have hold opens.

A plurality of openings (i.e., inlets, which are not shown but similar to the openings 72 of FIG. 5) are associate with each of the passages 568A, 568B for allowing the heat transfer gas to pass from the passage and into direct contact with the portion of the patient's body received in the interior space 516 of the enclosure 214. The openings in the base 524 allow heat transfer gas to pass to the porous layer 550, which is situated between the bottom, vapor impermeable member 548 and the portion of the patient's body. The number and location of openings positioned in various portions of the enclosure 514 may be varied to regulate the distribution of heat transfer gas throughout the enclosure. In one suitable configuration, the openings are positioned for evenly distributing the heat transfer gas throughout the enclosure 514. However, it is understood that the openings may be positioned to distribute heat transfer gas unevenly to the enclosure 514. By having an uneven flow distribution, a greater volume of heat transfer gas can be directed to selected portions of the patient's body, such as those more amenable to heat transfer (i.e., the head, neck, torso) than other non-selected portions of the patient's body, which are also received in the enclosure 514.

Both the blanket 522 and the base 524 also include a plurality of outlet passages 569A, 569B configured to allow heat transfer gas to exit the enclosure 514. In one suitable embodiment, each of the outlet passages 569A, 568B formed in both the blanket 522 and the base 524 are supported by a hold-open 571, which holds the passage open and permits flow of the heat transfer gas through the passage past the hold-open. A plurality of openings (i.e., outlets, which are not shown but similar to the openings 72 of FIG. 5) are associated with each of the passages 569A, 569B for allowing the heat transfer gas to pass from the interior space 516 of the enclosure 214 and into the respective passage 569A, 569B.

Referring again to FIG. 18, the gas body temperature heating/cooling apparatus 500 further comprises a control system, generally indicated at 600, for controlling operation of the apparatus. The control system 600, which in the illustrated embodiment is adapted to hang from a conventional IV pole 698, includes a controller 602, a monitor 604 (broadly, a “user interface”), a gas delivery system, and a temperature sensor 608 for measuring the temperature of the patient P. The monitor 604 includes an LCD touch screen display for visually indicating particular parameters of the control system 600 and for allowing the user of the system to selectively control particular system functions. The monitor 604, for example, could display a target temperature along with the actual body temperature of the patient P, and the temperature of the heat transfer gas, among other things. In one suitable embodiment, the monitor 604 is similar to the display 94 illustrated in FIG. 10A and described above. With respect to user control of the system 600, the user can start, pause, and stop the delivery system using the touch screen display of the monitor 604. It is also understood that other system 600 functions could be controlled by the user using the touch screen display of the monitor 604.

It is understood that the control system 600 can have other suitable embodiments. In one embodiment, the control system 600 can be substantially the same as the controller 86 illustrated in FIGS. 10 and 10A. In another embodiment, the control system 600 can be substantially the same as the control system 300 illustrated in FIG. 18. In this embodiment, the same control system 300 can be adapted to control both the first, liquid body temperature cooling/heating apparatus 200 and the second, gas body temperature cooling/heating apparatus 500 of the system 175. It is also understood that the control system 600 can be adapted to be mounted on other suitable structure, e.g., a mobile cart (similar to controller system 300), the gurney G, and/or a hospital bed.

In one embodiment, the gas delivery system is a generally closed, continuous flow system in which heat transfer gas exhausted from the enclosure 214 via the outlet passages 569A, 569B is directed to flow back to enclosure via the inlet passages 568A, 568B and into the interior space 516 of the enclosure for direct contact with the patient's body. The gas delivery system comprises a gas heat exchanger 618, an air pump 630, and a filtration system 612. It is understood that the gas delivery system can have fewer or more components without departing from the scope of this invention.

In one suitable embodiment, the gas delivery system may include a humidity adjustment unit for increasing humidity or reducing humidity in the gas being delivered to the interior space 516 of the enclosure 514. The reduction of humidity in the gas may help keep the skin of the patient dry and thereby protect against decubitus ulcers. Humidity may be added to the gas to increase the heat exchange rate between the patient P and the gas or to prevent desiccation of compromised tissues.

An exhaust pump 621 may also be added to the gas control system for drawing heat transfer gas from the interior space 516 of the enclosure 514 by applying a vacuum thereto. The hold-opens 571 provided in the outlet passages 569A, 569B inhibit the passages from collapsing under the influence of the vacuum applied by the exhaust pump 621. The exhaust pump 621 can drive the exhaust heat transfer gas through the filtration system 612. In one suitable configuration, the exhaust pump 621 draws heat transfer gas from the interior space 516 of the enclosure 514 at a rate greater than the rate at which the air pump 630 is introducing heat transfer gas into the interior space to create a negative pressure environment within the enclosure. The negative pressure environment would prevent heat transfer gas and any potential undesirable airborne agents (e.g., viruses, bacteria, noxious gases) from escaping the enclosure 514.

The air pump 630, such as a conventional reciprocating or scroll-type compressor, is in fluid communication to pump gas into the enclosure 514 for heat transfer purposes. The gas heat exchanger 618, such as an inline air heater (e.g., electrical resistance heater) or cooler (e.g., an air conditioner), can be used to alter the temperature of the heat transfer gas prior to it being pumped into the interior space 516 of the enclosure 514. Accordingly, the temperature altered gas can be directed into the enclosure 516 to maintain the temperature of the patient received in the enclosure at or near the target temperature. The temperature altered gas can be used to alter (cool or warm) the temperature of the patient P. In one suitable embodiment, if the patient's body temperature exceeds 33.5° C. cooled gas can be delivered to the patient (broadly, a gas cooling mode of the system) and if the patient's body temperature drops below 32.5° C. warmed gas be delivered to the patient (broadly, a gas warming mode of the system). In one embodiment, the control system 600 can be used to selectively adjust the temperature of the gas and the rate at which the gas is delivered to the interior space 516 of the enclosure 514.

The filtration system 612 is in fluid communication with the outlet passages 569A, 569B for filtering the heat transfer gas as it is exhausted from the enclosure 514. The filtration system 612 can comprises a particular matter filter, activated carbon, and/or an ultraviolet light to kill bacteria and viruses. The filtration system 612 can be selected to prevent the spread of one or more airborne agent (i.e., viruses, bacteria, noxious gases) to the surrounding environment. The filtration system 612 can be located anywhere within the gas delivery system or have more or fewer filtration capabilities without departing from the scope of this invention.

An umbilicus, indicated generally at 620, is used to simply and easily connect the air pump 630 to the enclosure 514. The umbilicus 620 includes two flexible air supply conduits 549 for supplying air from the air pump 630 to the inlet passages 568A, 568B in both the blanket 522 and the base 524. Specifically, one of the air supply conduits 568A feeds the inlet passages 568A in the blanket 522 and the other air supply conduit feeds inlet passages 568B the base 242. The umbilicus 620 further includes the return conduit 582 that feeds heat transfer gas from the enclosure 514 via the outlet passages 569A, 569B in the blanket 522 and base 524 through the filter system 612. If the exhaust pump 621 is used, then heat transfer gas is drawn from the interior space 516 of the enclosure 514 by the exhaust pump through the return conduit 582 and through the filter system 612. Each end of the umbilicus 620 comprises a quick-connect coupling 660 (one being shown in FIG. 18) to attach the ends of the umbilicus to the control system 600 and the enclosure 514 to establish a fluid connect therebetween.

It is contemplated that in some embodiments of the system 175, the second, gas cooling/heating apparatus 500 can comprise an conventional warming/cooling apparatus, such as, for example, the Gaymar Thermacare® system, the Cincinnati Sub-Zero patient temperature management systems, or the Medivance Arctic Sun system.

In operation, the enclosure 214 of the first, liquid cooling/heating apparatus 200 is placed in an uninflated state on a generally flat surface, such the ambulance gurney G. The compliant support 224 is fully extended to a position such that the underside of the compliant support is resting on the gurney G. If not already done, the cover 222 is removed from the compliant support 224 by disengaging the sealing portions 294 to expose the center of the compliant support 224. The patient P is carefully placed on the base 242 of the compliant support 224. Using the touch screen display on the monitor 304, the user activates the inflating system via the controller 302. In response, the controller 302 activates the air pump 316 to inflate the tubes 244A, 244B and the base 242 to the desired pressure. As explained above, inflating the tubes 244A, 244B and the base 242 conforms the well of the complaint support 224 to the portion of the patient's body received therein.

The cover 222 is placed on the patient P to cover the patient's body from the neck downward. The sealing portions 294 of the cover 222 and the compliant support 224 are engaged thereby enclosing the patient P in the interior space 216 of the enclosure 214. The temperature sensor 308 (i.e., thermometer) is connected to the patient P for measuring the core body temperature of the patient. The temperature sensor 308 is also connected to the controller 302 so that the measured body temperature of the patient P can be conveyed to the controller.

The reservoir 312 is filled with the appropriate amount of ice (or other phase change material) and heat transfer liquid. Using the touch screen display on the monitor 304, the liquid delivery system can be activated. Once activated, the liquid inlet pump 314 delivers heat transfer liquid to the patient's body to adjust the temperature of the patient P to a selected temperature. For example, it may be desirable to rapidly lower the body temperature of a patient P suffering from cardiac arrest from about 37° C. (98.6° F.) to about 33° C. (91.4° F.).

In one example, approximately 30 liters (8 gallons) of the heat transfer liquid (e.g., water) and approximately 10 kilograms (22 pounds) of phase change material (e.g., ice) can be added to the reservoir 312. The heat transfer liquid, which is lowered to a temperature between about 0° C. (32° F.) and about 5° C. (41° F.), is drawn from the reservoir 312 by the liquid inlet pump 314 and pumped through umbilicus 320 and into the passages in the cover 222 and the compliant support 224 and thereby into the top and bottom of the interior space 216 of the enclosure 214.

The heat transfer liquid is directed back into the reservoir 312 through the drain tube 282 of the umbilicus 320 where it is re-cooled by the phase change material before being recirculated back into the interior space 216 of the enclosure 214. Heat transfer liquid is continuously recirculated through the enclosure 214 until the patient's temperature reaches or approaches the selected temperature. The patient's temperature may drop slightly after the heat transfer liquid has been stopped and, as a result, it may be desirable to stop the flow of heat transfer liquid before the patient's temperature drops to the selected temperature to prevent overshoot (i.e., lowering the patient's body temperature below the selected temperature). For example, the controller 302 can be programmed to shut off the liquid delivery system when the core body temperature of the patient is within 1° C. or 2° C. of the target temperature to prevent the patient's core body temperature from falling below the target temperature. In addition, the controller 302 can be programmed to send a warning (i.e., an audio or visual alarm) to a user if the core body temperature falls below the target temperature.

Once the temperature of the patient P has reached the predetermined temperature (e.g., 1° C. or 2° C. above of the target temperature), the liquid inlet pump 314 is automatically shut off by the controller 302 and the heat transfer liquid is purged from the enclosure 214. In one configuration, the interior space 216 of the enclosure 214 can be purged by allowing any heat transfer liquid present in the interior space to flow via gravity through the drain tube 282 and back into the reservoir 312.

The inflatable tubes 244A, 244B and base 242 of the compliant support 224 can be deflated by activating one or more air release valves 278. In the illustrated configuration, the air release valves 278 comprise capped plugs that can be activated by manually removing the cap from the plug housing. It is to be understood that the other types of air release valves including automated valves can be used.

The patient P can be removed from the first, liquid cooling/heating apparatus 200 for a period of time without the patient's body temperature deviating much from the target temperature. That is, the patient's body will take some time before it begins to re-warm itself. In some cases, the patient's body temperature will remain at approximately the target temperature for up to six hours or longer. During this period of time, medical testing, examination, and treatments can be conducted on the patient P.

After removal from the first, liquid cooling/heating apparatus, the enclosure 514 of the second, a second, gas cooling/heating apparatus is placed on a generally flat surface, such the ambulance gurney G. The base 524 is fully extended to a position such that the underside of the base is resting on the gurney. The blanket 522 is disengaged from the base 524, if necessary. The patient P is carefully placed in the center of the base 524 on the porous layer 550. The patient P can be maintained at the target temperature by turning on the air pump 630 and directing the air pumped by the air pump through the gas heat exchanger 618 to cool the air to a desired temperature. The cooled heat transfer gas is directed to flow into the inlet passages 568A, 568B, through the associated openings, and into the interior space 516 of the enclosure 514 for direct contact with the patient's body. It is believed that the patient's body temperature can be maintained using heat transfer gas for an extended period of time (e.g., 1-3 days). It is understood that the patient's body temperature can be maintained at approximately the target temperature for any desired period of time. The heat transfer gas exits the interior space 516 of the enclosure 514 through the outlet passages 569A, 569B were it is passes through the filtration system 612.

After the patient P has been maintained at the target temperature of the desired period of time, the patient's body temperature can be re-warmed to approximately its normal temperature (i.e., about 37° C.). To re-warm the patient P, the air pump 630 is turned on and air is pump through the gas heat exchanger 618 to warm the air to a desired temperature (broadly, a re-warming mode of the system). The warmed heat transfer gas is directed to flow into the inlet passages 568A, 568B, through the associated openings, and into the interior space 516 of the enclosure 514 for direct contact with the patient's body. In one suitable embodiment, the patient's body temperature can be re-warmed using the heat transfer gas at a rate of approximately 0.2° C./hour to approximately 0.5° C./hour but it is understood that faster or slower rates can be used. The heat transfer gas exits the interior space 516 of the enclosure 514 through the outlet passages 569A, 569B where it passes through the filtration system 612.

Once the patient's body has reached approximately its normal body temperature, the patient can be removed from the second, gas cooling/heating apparatus.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

What is claimed is:

1. A system comprising:
a temperature reducing apparatus comprising a first enclosure defining an interior space for receiving at least a portion of a patient's body therein, and a liquid delivery system in fluid communication with the first enclosure for controlling the temperature of a heat transfer liquid and delivering the heat transfer liquid into the first enclosure in direct contact with the patient's body portion when received in the first enclosure; and
a temperature maintenance apparatus comprising a second enclosure defining an interior space for receiving at least a portion of a patient's body therein, and a gas delivery system in fluid communication with the second enclosure for controlling the temperature of a heat transfer gas and delivering the heat transfer gas into the second enclosure in direct contact with the patient's body portion when received in the second enclosure, the second enclosure being different than the first enclosure.

2. The system as set forth in claim 1 further comprising at least one control system for operating both the temperature reducing apparatus and the temperature maintenance apparatus.

3. The system as set forth in claim 2 wherein the system includes a first control system for operating the temperature reducing apparatus and a second control system for operating the temperature maintenance apparatus.

4. The system as set forth in claim 2 further comprising a sensor for measuring a body temperature of the patient, the sensor being adapted for communicating with the control system, the control system including a display capable of displaying the temperature of the patient.

5. The system as set forth in claim 4 wherein the display is adapted to indicate a directed readout of the patient temperature.

6. The system as set forth in claim 4 wherein the display is adapted to indicate the temperature of the patient graphically as a function of time.

7. The system as set forth in claim 4 wherein the control system includes at least one warning indicator adapted to signal when the patient's temperature deviates too far from the target temperature.

8. The system as set forth in claim 7 wherein the warning indicator includes a red light that illuminates when the patient's temperature deviates too far above the target temperature, and a blue light that illuminates when the patient's temperature deviates too far below the target temperature.

9. The system as set forth in claim 8 wherein the warning indicator further includes an audible warning signal.

10. The system as set forth in claim 1 wherein the gas delivery system includes a filtration system for filtering the heat transfer gas after the heat transfer gas has passed through the second enclosure and into direct contact with the patient's body portion when received in the second enclosure.

11. A system comprising:
a temperature reducing apparatus comprising a first enclosure defining an interior space for receiving at least a portion of a patient's body therein, and a liquid delivery system in fluid communication with the first enclosure for controlling the temperature of a heat transfer liquid and delivering the heat transfer liquid into the first enclosure in direct contact with the patient's body portion when received in the first enclosure; and
a temperature maintenance and re-warming apparatus comprising a second enclosure defining an interior space for receiving at least a portion of a patient's body therein, and a gas delivery system in fluid communication with the second enclosure for controlling the temperature of a heat transfer gas and delivering the heat transfer gas into the second enclosure in direct contact with the patient's body portion when received in the second enclosure, the delivery system having a heat exchanger capable of warming the heat transfer gas and cooling the heat transfer gas.

12. The system as set forth in claim 11 further comprising at least one control system for operating the temperature reducing apparatus and the temperature maintenance and re-warming apparatus.

13. The system as set forth in claim 12 wherein the system includes a first control system for operating the temperature reducing apparatus and a second control system for operating the temperature maintenance and re-warming apparatus.

14. The system as set forth in claim 11 wherein the first enclosure comprises a compliant support adapted to underlie and generally conform to the shape of the portion of the patient's body, and a cover for covering the portion of the patient's body.

15. The system as set forth in claim 11 wherein the second enclosure comprises a blanket for overlying the portion of the patient's body.

16. The system as set forth in claim 12 wherein the second enclosure further comprises a base for underlying the portion of the patient's body.

17. The system as set forth in claim 16 wherein the base comprises a porous layer capable of allowing air to pass therethrough and into contact the portion of the patient's body.

18. The system as set forth in claim 17 wherein the base further comprises at least one hold-open associated with the base.

19. The system as set forth in claim 16 wherein the gas delivery system includes a filtration system for filtering the heat transfer gas after the heat transfer gas has passed through the second enclosure and into direct contact with the patient's body portion when received in the second enclosure.

20. The system as set forth in claim 16 wherein gas delivery system further includes an exhaust pump in fluid communication with the second enclosure and the filtration system for drawing heat transfer gas from the interior space of the second enclosure and driving it through the filtration system.

21. A method for operating a system adapted to adjust the body temperature of a patient, the method comprising:
enclosing at least a portion of a patient's body within an interior space of a first enclosure, the first enclosure having an inlet for receiving a heat transfer liquid into the interior space, and an outlet in fluid communication with the interior space of the enclosure for exhausting the heat transfer liquid from the enclosure;
directing the heat transfer liquid through the inlet of the enclosure into the interior space for flow over the patient's body in direct liquid contact therewith to promote heat transfer between the patient's body and the heat transfer liquid to the outlet of the enclosure;
removing the portion of the patient's body from the first enclosure;
enclosing the portion of the patient's body within an interior space of a second enclosure; and
directing a heat transfer gas into the interior space of the second enclosure for flow over the patient's body in direct contact therewith to promote heat transfer between the patient's body and the heat transfer gas.

22. The method as set forth in claim 21 further wherein directing heat transfer liquid into the interior space of the first enclosure is performed to alter the body temperature of the patient to approximately a target temperature.

23. The method as set forth in claim 22 wherein directing heat transfer gas into the interior space of the second enclosure is performed to maintain the body temperature of the patient generally at the target temperature for a predetermined period of time.

24. The method as set forth in claim 23 further comprising directing heat transfer gas into the interior space of the second enclosure to re-warm the patient to approximately a normal temperature of the patient after the predetermined period of time has passed.

25. The method as set forth in claim 22 wherein more than fifteen minutes passes before the portion of the patient's body is enclosed within an interior space of a second enclosure after the portion of the patient's body is removed from the first enclosure.

26. A gas body temperature cooling/heating apparatus comprises:
an enclosure defining an interior space for receiving at least a portion of a patient's body, the enclosure being adapted to allow heat transfer gas to flow into the interior space for direct contact with the patient's body to promote heat transfer between the patient and the heat transfer gas;
a gas delivery system having a gas heat exchanger for controlling the temperature of the heat transfer gas, and an exhaust pump for drawing heat transfer gas from the interior space of the enclosure and creating a vacuum within the interior space of the enclosure.

27. The gas body temperature cooling/heating apparatus as set forth in claim 26 further comprising a filtration system for filtering the heat transfer gas as it is exhausted from the interior space of the enclosure.

28. The gas body temperature cooling/heating apparatus as set forth in claim 26 wherein the gas delivery system further includes an air pump for directing the heat transfer gas into the interior space of the enclosure for direct contact with the patient's body, the exhaust pump being adapted to draw heat transfer gas from the interior space of the enclosure at a rate greater than the rate at which the air pump is adapted to introduce heat transfer gas into the interior space.

29. The gas body temperature cooling/heating apparatus as set forth in claim 26 wherein the gas delivery system further includes a humidity adjustment unit for increasing humidity or reducing humidity in the gas being delivered to the interior space of the enclosure.

30. The gas body temperature cooling/heating apparatus as set forth in claim 26 wherein the enclosure comprises a blanket for overlying the patient from the neck downward.

31. The gas body temperature cooling/heating apparatus as set forth in claim 30 wherein the enclosure further comprises a base for underlying the patient's entire body.

32. The gas body temperature cooling/heating apparatus as set forth in claim 31 wherein the base of the enclosure includes a bottom, vapor impermeable sheet-like member and a porous layer overlying the member.

33. The gas body temperature cooling/heating apparatus as set forth in claim 31 wherein the blanket and the base are adapted for sealing engagement with each other.
